(19) United States
(12) Patent Application Publication
(10) Pub. No.: US 2009/0181934 A1
(43) Pub. Date: Jul. 16, 2009

(54) Organic Compounds

(75) Inventor: Robin Alec FAIRHURST, Horsham (GB)

Correspondence Address: NOVARTIS INSTITUTES FOR BIOMEDICAL RESEARCH, INC., 220 MASSACHUSETTS AVENUE, CAMBRIDGE, MA, 02139, US

(73) Assignee: Novartis AG

(21) Appl. No.: 12/247,764

(22) Filed: Oct. 8, 2008

(30) Foreign Application Priority Data
Oct. 17, 2007 (EP) 07118721.5

(51) Int. Cl. A61K 31/58; A61K 31/56; A61P 11/00; A61K 31/565; A61K 31/52

(52) U.S. Cl. 514/171; 514/263.22; 514/263.21

(57) ABSTRACT

A composition comprising, separately or together: a component (A) that is an adenosine A₂ₐ receptor agonist as defined in the specification; and a component (B) that is one or more compounds selected from: (i) a corticosteroid, (ii) a beta-2 adrenoceptor agonist, (iii) an antimuscarinic agent, (iv) an A_{2B} antagonist, (v) an antihistamine, (vi) a caspase inhibitor, (vii) an ENaC inhibitor, (viii) an LTB4 antagonist, (ix) an LTD4 antagonist, (x) a serine protease inhibitor, (xi) a PDE4 inhibitor and (xii) a dual-acting beta-2 adrenoceptor agonist/muscarinic antagonist, for simultaneous, sequential or separate administration in the treatment of an inflammatory or obstructive airways disease.

This application claims priority to E.P. Application Serial No. 07118721.5 filed 17 Oct. 2007, the contents of which are incorporated herein by reference in their entirety.

This invention relates to organic compounds and their use as pharmaceuticals, in particular for the treatment of inflammatory or obstructive airways diseases.

In one aspect, the present invention provides a medicament comprising, separately or together

(A) a compound selected from:
((1S,2R,3S,4R)-4-{6-(2,2-Diphenyl-ethylamino)-2-[(S)-3-(3-pyridin-3-yl-ureido)-pyrrolidin-1-yl]-purin-9-yl}-2,3-dihydroxy-cyclopentyl)-carbamic acid methyl ester;
((1S,2R,3S,4R)-4-{6-(2,2-diphenyl-ethylamino)-2-[(R)-3-(3-pyridin-3-yl-ureido)-pyrrolidin-1-yl]-purin-9-yl}-2,3-dihydroxy-cyclopentyl)-carbamic acid methyl ester;
cyclopropanecarboxylic acid ((1S,2R,3S,4R)-4-{6-(2,2-diphenyl-ethylamino)-2-[(R)-3-(3-pyridin-3-yl-ureido)-pyrrolidin-1-yl]-purin-9-yl}-2,3-dihydroxy-cyclopentyl)-amide;
N-((1S,2R,3S,4R)-4-{6-(2,2-diphenyl-ethylamino)-2-[(R)-3-(3-pyridin-2-ylmethyl-ureido)-pyrrolidin-1-yl]-purin-9-yl}-2,3-dihydroxy-cyclopentyl)-propionamide trifluoroacetate;
N-[(1S,2R,3S,4R)-4-(6-((S)-1-benzyl-2-hydroxy-ethylamino)-2-{(R)-3-[3-(4-sulfamoyl-phenyl)-ureido]-pyrrolidin-1-yl}-purin-9-yl)-2,3-dihydroxy-cyclopentyl]-2-hydroxy-acetamide;
[(1S,2R,3S,4R)-4-(6-(2,2-diphenyl-ethylamino)-2-{(R)-3-[3-(3-sulfamoyl-phenyl)-ureido]-pyrrolidin-1-yl}-purin-9-yl)-2,3-dihydroxy-cyclopentyl]-carbamic acid methyl ester;
N,N-(1S,1S′,2R,2R′,3S,3S′,4R,4R′)-4,4′-((S)-2,2′-((3R,3′R)-3,3′-carbonylbis(azanediyl)bis(pyrrolidine-3,1-diyl))bis(6-((S)-1-hydroxy-3-phenylpropan-2-ylamino)-9H-purine-9,2diyl))bis(2,3-dihydroxycyclopentane-4,1-diyl)bis(2-hydroxyacetamide); and
N,N-(1S,1S′,2R,2R′,3S,3S′,4R,4R′)-4,4′-(6,6-(1R,4R)-cyclohexane-1,4-diylbis(azanediyl)bis(2-(2-(1-methyl-1 Himidazol-4-yl)ethylamino)-9H-purine-9,6-diyl))bis(2,3-dihydroxycyclopentane-4,1-diyl)bis(2-hydroxyacetamide),
   in free or salt or solvate form; and
   (B) one or more compounds selected from:

(i) a corticosteroid,

(ii) a beta-2 adrenoceptor agonist,

(iii) an antimuscarinic agent,

(iv) an A_{2B} antagonist,

(v) an antihistamine,

(vi) a caspase inhibitor,

(vii) an ENaC inhibitor,

(viii) an LTB4 antagonist,

(ix) an LTD4 antagonist,

(x) a serine protease inhibitor,

(xi) a PDE4 inhibitor, and

(xii) a dual-acting beta-2 adrenoceptor agonist/muscarinic antagonist;

for simultaneous, sequential or separate administration in the treatment of an inflammatory or obstructive airways disease.

In another aspect, the present invention provides a pharmaceutical composition comprising a mixture of effective amounts of (A) as hereinbefore defined and (B) as hereinbefore defined, optionally together with at least one pharmaceutically acceptable carrier.

In a further aspect, the present invention provides a method of treating an inflammatory or obstructive airways disease or diseases associated with the regulation of fluid loss across epithelial membranes which comprises administering to a subject in need of such treatment effective amounts of (A) as hereinbefore defined and (B) as hereinbefore defined.

The invention further provides the use of (A) as hereinbefore defined and (B) as hereinbefore defined in the preparation of a medicament for combination therapy by simultaneous, sequential or separate administration of (A) and (B) in the treatment of an inflammatory or obstructive airways disease.

In an embodiment of the invention as defined above, the corticosteroid (B)(i) may be GSK685698, GSK870086, or for example, be a compound of formula X

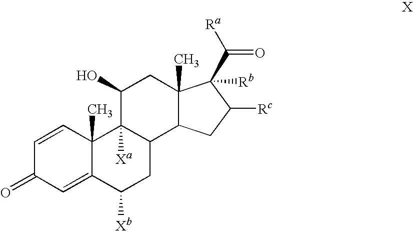

or a 1,2-dihydro derivative thereof, where
R^{a} is C₁-C₄-alkyl optionally substituted by halogen (such as chlorine or fluorine), hydroxy, C₁-C₄-alkoxy, acyloxy or by C₁-C₄-acylthio, or R^{a} is C₁-C₄-alkoxy or C₁-C₄-alkylthio optionally substituted by halogen, or R^{a} is 5- or 6-membered heterocyclylthio, or R^{a} is C₁-C₄-alkylthio optionally substituted by halogen (such as chlorine or fluorine),
   either R^{b} is acyloxy and R^{c} is hydrogen or C₁-C₄-alkyl,
   or R^{b} and R^{c} together denote a group of formula XI

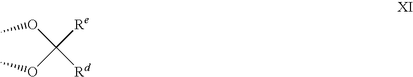

where R^{d} is C₁-C₄-alkyl or C₃-C₆-cycloalkyl and R^{e} is hydrogen or C₁-C₄-alkyl, X^{a} and X^{b} are each independently hydrogen, chlorine or fluorine.

When R^{a} is acyloxy-substituted C₁-C₄-alkyl, the acyloxy group may be, for example, C₁-C₂₀-alkylcarbonlyloxy, e.g. acetyloxy, n-propionyloxy, isopropionyloxy or hexadecanoyloxy, or C₃-C₆-cycloalkylcarbonyloxy, e.g. cyclohexylcarbonyloxy. When R^{a} is acylthio-substituted C₁-C₄-alkyl, the acylthio group may be, for example, C₁-C₄-alkylcarbonylthio, e.g. acetylthio or n-propionylthio. When R^{a} is 5- or 6-membered heterocyclylthio, the heterocyclyl group may be an O-heterocyclyl group, for example a furanonyl group.

When R^{b} is acyloxy, it may be, for example, C₁-C₄-alkylcarbonyloxy, e.g. acetyloxy, n-propionyloxy, or n-butyryloxy, C₁-C₄-cycloalkylcarbonyloxy e.g. cyclopropylcarbonyloxy, or 5- or 6-membered heterocyclylcarbonyloxy e.g. furoyloxy, or when R^{b} is acyloxy it may be a group —O—CO-T where T is a monovalent cyclic organic group having from 3 to 15 atoms in the ring system. Suitably T is a carbocyclic group or a heterocyclic group having one or more ring hetero atoms selected from nitrogen, oxygen and sulfur.

When R^{c} is C₁-C₄-alkyl it may be in the alpha or beta conformation, more usually in the alpha conformation.

When R^{b} and R^{c} together denote a group of formula XI, R^{d} as C₃-C₆-cycloalkyl may be, for example, cyclohexyl.

Corticosteroids of formula X and their 1,2-dihydro derivatives include beclamethasone dipropionate, budesonide, fluticasone propionate, mometasone furoate, ciclesonide, triamcinolone acetonide, flunisolide, rofleponide palmitate, butixocort propionate, icometasone enbutate and described in WO 03/042229, WO 03/035668, WO 02/100879, WO 02/088167.

In a further embodiment of the invention as defined anywhere above, the corticosteroid (B)(i) is budesonide, fluticasone propionate, mometasone furoate or either of the following compounds:

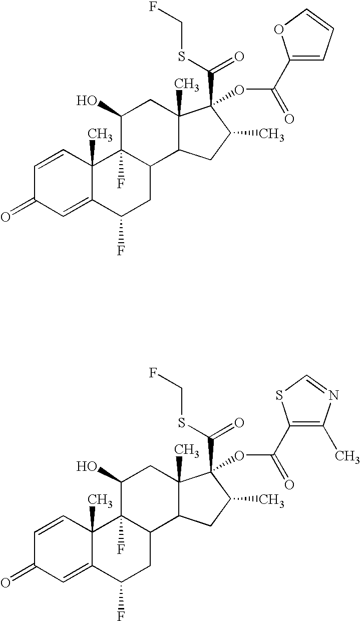

Budesonide, fluticasone propionate and mometasone furoate and their preparation are described in U.S. Pat. No. 3,929,768, U.S. Pat. No. 4,335,121 and U.S. Pat. No. 4,472,393 respectively.

Corticosteroids of formula X where R^{b} is —O—CO-T are suitably compounds of formula XII

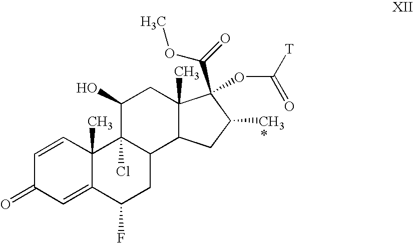

where T is a monovalent cyclic organic group having from 3 to 15 atoms in the ring system.

Suitably T is a carbocyclic group or a heterocyclic group having one or more ring hetero atoms selected from nitrogen, oxygen and sulfur.

In an embodiment of the invention as defined anywhere above, T is a cycloaliphatic group having 3 to 8 carbon atoms, for example C₃-C₈-cycloalkyl such as cyclopropyl, methylcyclopropyl, cyclobutyl, methylcyclobutyl, cyclopentyl, cyclohexyl, methylcyclohexyl, dimethylcyclohexyl or cycloheptyl, suitably C₃-C₆-cycloalkyl.

In another embodiment, T is an at least partially saturated heterocyclic group having 5 to 10 ring atoms, of which one or more are ring hetero atoms selected from nitrogen, oxygen and sulfur, optionally having 5 to 7 ring atoms, of which one or two are hetero atoms selected from nitrogen and oxygen, especially a 5-membered heterocyclic group having one ring hetero atom, such as a tetrahydrofuryl or oxotetrahydrofuryl group.

In a further embodiment, T is a carbocyclic or heterocyclic aromatic group having 5 to 15 atoms in the ring system. For example, T may be such an aromatic group in which the ring system is unsubstituted or is substituted by one or more substituents selected from halogen, cyano, C₁-C₄-alkyl, halo-C₁-C₄-alkyl, C₁-C₄-alkoxy, C₁-C₄-alkylthio, hydroxyl, C₁-C₄-acyl, C₁-C₄-acyloxy, amino, C₁-C₄ alkylamino, di-(C₁-C₄-alkyl)amino, C₁-C₄-acylamino, C₁-C₄-acyl(C₁-C₄-alkyl)-amino, C₁-C₄-alkylsulfonyl(C₁-C₄-alkyl)amino, C₁-C₄-alkoxycarbonyl, or 5-membered heterocyclyl, usually N-heterocyclyl having one or two nitrogen atoms. One suitable class of such aromatic groups is phenyl or naphthyl optionally substituted by one or more, suitably one, two or three, substituents selected from cyano, C₁-C₄-alkyl, halo-C₁-C₄-alkyl, C₁-C₄ alkoxy, halogen, hydroxyl, C₁-C₄-acyloxy, amino, C₁-C₄-alkylamino, di-C₁-C₄-alkylamino, C₁-C₄-acyl-amino, C₁-C₄-acyl(C₁-C₄ alkyl)amino, C₁-C₄ alkylsulfonyl(C₁-C₄ alkyl)amino or C₁-C₄-alkoxy-carbonyl, especially suitable aromatic groups including phenyl, cyanophenyl, tolyl, dimethylphenyl, ethylphenyl, (trifluoromethyl)phenyl, dimethoxy-phenyl, diethoxyphenyl, hydroxyphenyl, (methylamino)phenyl, (methanesulfonylmethylamino)-phenyl and (methoxy-carbonyl)phenyl.

Another suitable class of such aromatic groups is a heterocyclic aromatic group having a 6-membered heterocyclic ring with one, two or three ring heteroatoms, suitably nitrogen, the heterocyclic ring being unsubstituted or substituted by one or more, preferably one, two or three, substituents selected from halogen, cyano, hydroxyl, C₁-C₄-acyloxy, amino, C₁-C₄-alkyl-amino, di-(C₁-C₄-alkyl)amino, C₁-C₄-alkyl, hydroxy-C₁-C₄-alkyl, halo-C₁-C₄-alkyl, C₁-C₄-alkoxy, or C₁-C₄-alkylthio, and the heterocyclic ring being optionally fused to a benzene ring. Suitable heterocyclic aromatic groups include those in which the heterocyclic group has one or two nitrogen atoms in the ring, especially a pyridine, pyrimidine, pyrazine or pyridazine ring. Especially suitable heterocyclic aromatic groups are pyridyl, pyrimidinyl and pyrazinyl groups, optionally substituted by one or two substituents selected from halogen (particularly chlorine) or C₁-C₄-alkyl (especially methyl or n-butyl).

Another suitable class of such aromatic groups is a heterocyclic aromatic group having a 5-membered heterocyclic ring with one, two or three ring hetero atoms selected from nitrogen, oxygen and sulfur, the heterocyclic ring being unsubstituted or substituted by one or two substituents selected from halogen, C₁-C₄-alkyl, halo-C₁-C₄-alkyl, C₁-C₄-alkoxy, C₁-C₄-alkyl-thio, cyano or hydroxy-C₁-C₄-alkyl and the heterocyclic ring being optionally fused to a benzene ring. Specific examples of such heterocyclic aromatic groups include those in which the heterocyclic ring has one nitrogen, oxygen or sulfur atom in the ring or one oxygen and one or two nitrogen atoms in the ring, or one sulfur and one or two nitrogen atoms in the ring, especially a pyrrole, furan, thiophene, oxazole, isoxazole, imidazole, pyrazole, furazan, thiazole or thiadiazole ring. Especially suitable heterocyclic aromatic groups are pyrrolyl, furyl and thienyl groups optionally substituted by one or two substituents selected from halogen (particularly chlorine or bromine), C₁-C₄-alkyl (particularly methyl or ethyl), halo-C₁-C₄-alkyl (particularly trifluoro-methyl), C₁-C₄-alkoxy (particularly methoxy), C₁-C₄-alkylthio (particularly methylthio), cyano or hydroxy-C₁-C₄-alkyl (particularly hydroxymethyl); isoxazolyl, imidazolyl, pyrazolyl, thiazolyl or thiadiazolyl groups optionally substituted by one or two C₁-C₄-alkyl groups; and benzofuryl, benzothienyl and benzofurazanyl groups.

In compounds of formula XII, the indicated methyl group in the 16 position of the corticosteroid ring system may be in the alpha or beta conformation. 16-α-methyl compounds are particularly suitable.

Especially suitable compounds of formula XII are those where the indicated 16-methyl group has the alpha conformation and T is S-methyl-2-thienyl, N-methyl-2-pyrrolyl, cyclopropyl, 2-furyl, 3-methyl-2-furyl, 3-methyl-2-thienyl, 5-methyl-3-isoxazolyl, 3,5-dimethyl-2-thienyl, 2,5-dimethyl-3-furyl, 4-methyl-2-furyl, 4-(dimethylamino)phenyl, 4-methylphenyl, 4-ethylphenyl, 2-pyridyl, 4-pyrimidyl or S-methyl-2-pyrazinyl or the indicated 16-methyl group has the beta conformation and R is cyclopropyl.

The compounds of formula XII and salts thereof where T contains a basic group may be prepared using the procedures described in international patent application WO 02/00679.

The corticosteroid (B)(i) may, for example, also be a non-steroidal glucocorticoid receptor agonist, such as those described in DE 10261874, WO 00/00531, WO 02/10143, WO 03/82280, WO 03/82787, WO 03/86294, WO 03/104195, WO 03/101932, WO 04/05229, WO 04/18429, WO 04/19935 and WO 04/26248.

Terms used in the specification have the following meanings:

“Optionally substituted” as used herein means the group referred to can be substituted at one or more positions by any one or any combination of the radicals listed thereafter.

“Halo” or “halogen” as used herein denotes a element belonging to group 17 (formerly group VII) of the Periodic Table of Elements, which may be, for example, fluorine, chlorine, bromine or iodine. Preferably halo or halogen is fluorine or chlorine.

“C₁-C₄-alkyl” as used herein denotes straight chain or branched alkyl that contains one to four carbon atoms. If a different number of carbon atoms is specified, then the definition is to be construed accordingly.

“C₁-C₄-alkylene” as used herein denotes a straight chain or branched alkylene that contains one to four carbon atoms, suitably ethylene or methylethylene. If a different number of carbon atoms is specified, then the definition is to be construed accordingly.

“C₂-C₄-alkenyl” as used herein denotes straight chain or branched hydrocarbon chains that contain two to four carbon atoms and one or more carbon-carbon double bonds. If a different number of carbon atoms is specified, then the definition is to be construed accordingly.

“C₂-C₄-alkynyl” as used herein denotes straight chain or branched hydrocarbon chains that contain two to ten carbon atoms and one or more carbon-carbon triple bonds. If a different number of carbon atoms is specified, then the definition is to be construed accordingly.

“C₃-C₆-cycloalkyl” as used herein denotes cycloalkyl having 3 to 6 ring carbon atoms, for example a monocyclic group such as a cyclopropyl, cyclobutyl, cyclopentyl or cyclohexyl, any of which can be substituted by one or more, usually one or two, C₁-C₄-alkyl groups. If a different number of carbon atoms is specified, then the definition is to be construed accordingly.

“C₁-C₄-haloalkyl” as used herein denotes C₁-C₄-alkyl as hereinbefore defined substituted by one or more halogen atoms, preferably one, two or three halogen atoms. If a different number of carbon atoms is specified, then the definition is to be construed accordingly.

“C₁-C₄-alkylamino” and “di(C₁-C₄-alkyl)amino” as used herein denote amino substituted respectively by one or two C₁-C₄-alkyl groups as hereinbefore defined, which may be the same or different. If a different number of carbon atoms is specified, then the definition is to be construed accordingly.

“C₁-C₄-alkylthio” as used herein denotes straight chain or branched alkylthio having 1 to 4 carbon atoms. If a different number of carbon atoms is specified, then the definition is to be construed accordingly.

“C₁-C₄-alkoxy” as used herein denotes straight chain or branched alkoxy that contains 1 to 4 carbon atoms. If a different number of carbon atoms is specified, then the definition is to be construed accordingly.

“C₁-C₄-alkoxy-C₁-C₄-alkyl” as used herein denotes C₁-C₄-alkyl as hereinbefore defined substituted by C₁-C₄-alkoxy. If a different number of carbon atoms is specified, then the definition is to be construed accordingly.

“C₁-C₄-alkoxycarbonyl” as used herein denotes C₁-C₄-alkoxy as hereinbefore defined linked through an oxygen atom thereof to a carbonyl group. If a different number of carbon atoms is specified, then the definition is to be construed accordingly.

“C₆-C₁₀-aryl” as used herein denotes a monovalent carbocyclic aromatic group that contains 6 to 10 carbon atoms and which may be, for example, a monocyclic group such as phenyl or a bicyclic group such as naphthyl. Preferably C₆-C₁₀-aryl is C₆-C₈-aryl, especially phenyl. If a different number of carbon atoms is specified, then the definition is to be construed accordingly.

“C₆-C₁₀-arylsulfonyl” as used herein denotes C₆-C₁₀-aryl as hereinbefore defined linked through a carbon atom thereof to a sulfonyl group. Preferably C₆-C₁₀-arylsulfonyl is C₆-C₈-arylsulfonyl. If a different number of carbon atoms is specified, then the definition is to be construed accordingly.

“C₇-C₁₄-aralkyl” as used herein denotes alkyl, for example C₁-C₄-alkyl as hereinbefore defined, substituted by aryl, for example C₆-C₁₀-aryl as hereinbefore defined. Preferably, C₇-C₁₄-aralkyl is C₇-C₁₀-aralkyl such as phenyl-C₁-C₄-alkyl, particularly benzyl or 2-phenylethyl. If a different number of carbon atoms is specified, then the definition is to be construed accordingly.

“C₇-C₁₄-aralkyloxy” as used herein denotes alkoxy, for example C₁-C₄-alkoxy as hereinbefore defined, substituted by aryl, for example C₆-C₁₀-aryl. Preferably, C₇-C₁₄-aralkyloxy is C₇-C₁₀-aralkyloxy such as phenyl-C₁-C₄-alkoxy, particularly benzyloxy or 2-phenylethoxy. If a different number of carbon atoms is specified, then the definition is to be construed accordingly.

“Ar” or “aryl” as used herein may be, for example, phenylene which is unsubstituted or substituted by one or more substituents selected from halogen, hydroxy, C₁-C₄-alkyl, C₁-C₄-alkoxy, C₁-C₄-alkoxy-C₁-C₄-alkyl, phenyl, or C₁-C₄-alkyl substituted by phenyl, C₁-C₄-alkoxy substituted by phenyl, C₁-C₄-alkyl-substituted phenyl and C₁-C₄-alkoxy-substituted phenyl. Preferably Ar is phenylene which is unsubstituted or substituted by one or two substituents selected from halogen, C₁-C₄-alkyl, C₁-C₄-alkoxy, or C₁-C₄-alkoxy substituted by phenyl. If a different number of carbon atoms is specified, then the definition is to be construed accordingly.

“4- to 10-membered heterocyclic ring having at least one ring nitrogen, oxygen or sulphur atom” as used herein may be, for example, pyrrole, pyrrolidine, pyrazole, imidazole, triazole, tetrazole, thiadiazole, oxazole, isoxazole, thiophene, thiazole, isothiazole, oxadiazole, pyridine, pyrazine, pyridazine, pyrimidine, piperidine, piperazine, triazine, oxazine, morpholino, quinoline, isoquinoline, naphthyridine, indane or indene. Preferred heterocyclic rings include thiazole, pyrrolidine, piperidine, azacycloheptane and isoxazole. If a different number of ring atoms is specified, then the definition is to be construed accordingly.

“4 to 10-membered heterocyclyl-C₁-C₄-alkyl” denotes alkyl as hereinbefore defined, substituted by a 4- to 10-membered heterocyclic ring as hereinbefore defined. If a different number of carbon or ring atoms is specified, then the definition is to be construed accordingly.

“C₁-C₄-alkylsulfonyl” denotes sulfonyl substituted by C₁-C₄-alkyl as hereinbefore defined. If a different number of carbon atoms is specified, then the definition is to be construed accordingly.

“Hydroxy-C₁-C₄-alkyl” denotes C₁-C₄-alkyl as hereinbefore defined substituted by one or more, preferably one, two or three hydroxy groups. If a different number of carbon atoms is specified, then the definition is to be construed accordingly.

In an embodiment of the invention as defined above, the beta-2 adrenoceptor agonist (B)(ii) may, for example, be a compound which is a so-called long acting beta-2 adrenoceptor agonist (known commonly as a “LABA”). The ability of an agent to function as a beta-2 adrenoceptor agonist may be determined according to the methodologies disclosed by Battram et al Journal of Pharmacology and Experimental Therapeutics 2006, 317, 762-770.

Suitable beta-2 adrenoceptor agonists include albuterol (salbutamol), metaproterenol, terbutaline, salmeterol, fenoterol, procaterol, and especially, indacaterol, formoterol, carmoterol, milveterol, NVP-QAC455, GSIK159797, GSK159802, GSK597901, GSK642444, GSK678007 and pharmaceutically acceptable salts thereof, as well as those described in EP 147719, EP 1440966, EP 1460064, EP 1477167, EP 1574501, JP 05025045, JP 2005187357, US 2002/0055651, US 2004/0242622, US 2004/0229904, US 2005/0133417, US 2005/5159448, US 2005/5159448, US 2005/171147, US 2005/182091, US 2005/182092, US 2005/209227, US 2005/256115, US 2005/277632, US 2005/272769, US 2005/239778, US 2005/215542, US 2005/215590, US 2006/19991, US 2006/58530, US 2006/19991, US 2006/58530, WO 93/18007, WO 99/64035, WO 00/75114, WO 01/42193, WO 01/83462, WO 02/045703, WO 02/66422, WO 02/70490, WO 02/76933, WO 03/093219, WO 03/24439, WO 03/42160, WO 03/42164, WO 03/72539, WO 03/91204, WO 03/99764, WO 04/011416, WO 04/16578, WO 04/16601, WO 04/22547, WO 04/32921, WO 04/33412, WO 04/37768, WO 04/37773, WO 04/37807, WO 04/39762, WO 04/39766, WO 04/45618 WO 04/46083, WO 04/80964, WO 04/087142, WO 04/89892, WO 04/108675, WO 04/108676, WO 05/33121, WO 05/40103, WO 05/44787, WO 05/58867, WO 05/65650, WO 05/66140, WO 05/70908, WO 05/74924, WO 05/77361, WO 05/90288, WO 05/92860, WO 05/92887, WO 05/90287, WO 05/95328, WO 05/102350, WO 06/56471, WO 06/74897, WO 06/8173, WO 07/027,133, WO 07/027,134, WO 07/102,771 or WO 07/018,461.

An antimuscarinic agent is a substance or agent that inhibits acetyl choline binding to M3 muscarinic receptors thereby inhibiting bronchoconstriction. The ability of an agent to function as a muscarinic M3 antagonist may be determined according to the methodologies disclosed in international patent applications WO 06/048225. Suitable antimuscarinic agents include glycopyrrolate salts (particularly the bromide salt), ipratropium bromide, oxitropium bromide, tiotropium salts, (R)-3-(2-Hydroxy-2,2-diphenyl-acetoxy)-1-(isoxazol-3-ylcarbamoylmethyl)-1-azonia-bicyclo[2.2.2]octane, (R)-3-((R)-2-Cyclohexyl-2-hydroxy-2-phenyl-acetoxy)-1-(isoxazol-3-ylcarbamoylmethyl)-1-azonia-bicyclo[2.2.2]octane, CHF 4226 (Chiesi), GSK573719, GSK233705 and SVT-40776, or those described in EP 424021, U.S. Pat. No. 3,714,357, U.S. Pat. No. 5,171,744, US 2005/171147, US 2005/182091, WO 01/04118, WO 02/00652, WO 02/51841, WO 02/53564, WO 03/00840, WO 03/33495, WO 03/53966, WO 03/87094, WO 04/018422, WO 04/05285, WO 04/96800, WO 05/077361, WO 05/000815, WO 06/066928, WO 06/066929 and WO 06/48225.

Component (B) of the present invention optionally includes dual beta-2 adrenoceptor agonist/antimuscarinics such as biphenyl-2-yl-carbamic acid 1-(2-{(R)-3-[(R)-2-hydroxy-2-(4-hydroxy-2-oxo-2,3-dihydro-benzothiazol-7-yl)-ethylamino]-pyrrolidin-1-yl}-2-oxo-ethyl)-piperidin-4-yl ester, GSK961081 and those disclosed in US 2004/0167167, US 2004/0242622, US 2005/182092, US 2005/256114, US 2006/35933, WO 04/74246, WO 04/74812, WO 04/89892 and WO 06/23475.

An A_{2B} antagonist is a substance or agent that inhibits adenosine A_{2B} receptor activation. In general they selectively inhibit activation of the A_{2B} receptor over the adenosine A₁ and A_{2A} receptors. Their inhibitory properties may be demonstrated in the adenosine A_{2B} receptor reporter gene assay that is described in WO 02/42298. Suitable A_{2B} antagonists are described in WO 02/42298 and WO 03/042214.

Histamine is formed in vivo by the decarboxylation of histidine. It is released during allergic reactions such as hay fever and causes smooth muscle to contract and capillaries to dilate. Antihistamines inhibit the actions of histamine by blocking its site of action. Suitable antihistamine drug substances include cetirizine hydrochloride, levocetirizine, acetaminophen, clemastine fumarate, promethazine, loratidine, desloratidine, diphenhydramine and fexofenadine hydrochloride, activastine, astemizole, azelastine, dimetinden, ebastine, epinastine, levocabastine, mizolastine and tefenadine as well as those disclosed in JP 2004107299, WO 03/099807 and WO 04/026841.

A caspase inhibitor is a substance or agent that inhibits the activity of caspases, a family of enzymes involved in the induction of apoptosis in mammalian cells. The ability of an agent to function as a caspase inhibitor may be determined according to the methodologies disclosed in international patent applications WO 99/06367 and WO 99/65451. Suitable caspase inhibitors, including interleukin-I P converting enzyme (ICE) inhibitors, include those that are disclosed in Canadian patent specification 2109646 (para-nitroanilide peptides), European patent specification EP 519748 (peptidyl derivatives); EP 547 699 (peptidyl derivatives); EP 590 650 (cyclopropene derivatives); EP 628550 (pyridazines); EP 644 197 (peptidic phosphinyloxy-methyl ketones); EP 644198 (alpha-heteroaryloxymethyl ketones); international patent specification WO 93/05071 (peptidyl derivatives); WO 93/14777 (peptidyl derivatives); WO 93/16710 (peptidyl derivatives); WO 94/00154 (peptidyl derivatives); WO 94/03480 (peptidyl 4-amino-2,2-difluoro-3-oxo-1,6-hexanedioic acid derivatives); WO 94/21673 (alpha-keto-amide derivatives); WO 95/05152 (substituted ketone derivatives); WO 95/35308 (inhibitors comprising a hydrogen bonding group, a hydrophobic group and an electronegative group); WO 97/22618 (amino acid or di- or tripeptide amide derivatives); WO 97/22619 (N-acylamino compounds), WO 98-41232, WO 99/06367 (isatin sulphonamides); WO 99/65451, WO 01/119373, United States patent specification U.S. Pat. No. 5,411,985 (gamma-pyrone-3-acetic acid compounds); U.S. Pat. No. 5,416,013 (peptidyl derivatives); U.S. Pat. No. 5,430,128 (tripeptidyl derivatives); U.S. Pat. No. 5,434,248 (tripeptidyl compounds); U.S. Pat. No. 5,565,430 (N,N′-diacylhydrazinoacetic acid compounds); U.S. Pat. No. 5,585,357 (pyrazolyl derivatives); U.S. Pat. No. 5,656,627 (inhibitors comprising a hydrogen bonding group, a hydrophobic group and an electronegative group); U.S. Pat. No. 5,677,283 (pyrazolyl derivatives); U.S. Pat. No. 6,054,487, U.S. Pat. No. 6,531,474, US 20030096737 and United Kingdom patent specification GB 2,278,276 (gamma-pyrone-3-acetic compounds), as well as those disclosed in international patent applications WO 98/10778, WO 98/11109, WO 98/11129 and WO 03/32918.

An ENaC inhibitor is a substance or agent that inhibits the activity of epithelial sodium ion channels. These channels control the fluid that is absorbed into the bloodstream and thus regulate the airway surface liquid volume. If these channels are blocked in some way, fluid will collect in the lumen, which encourages mucus precursors to hydrate and stimulate mucus clearance. ENaC inhibitors can enhance mucus clearance and thus may be used to treat diseases associated with the impairment of mucociliary clearance. Pyrazinecarboxamides such as amiloride, benzamil and dimethyl-amiloride (DMA) are known to block human epithelial sodium channels. Amiloride has been used clinically as a diuretic but its short half life makes it unsuitable for use in treating airway disease. ENaC inhibitor activity cant be determined by measuring a change in transepithelial short circuit current using the method described by Baucher et al in Am. J. Respir. Crit. Care Med. 150: 221-281 (1994) or by using the assays described in WO 2002/087306 or WO 2004/72645. Suitable ENaC inhibitors include BAY39-9437, as well as those disclosed in international patent applications WO 07/071,400 and WO 07/071,396.

Leukotriene B4 antagonists inhibit the LTB4 receptor. Such compounds are useful in the treatment of conditions which respond to the inhibition of the LTB4 receptor, particularly inflammatory or allergic conditions. Suitable LTB4 antagonists include BIIL 284, CP-195543, DPC11870, LTB4 ethanolamide, LY 293111, LY 255283, CGS025019C, CP-195543, ONO-4057, SB 209247, SC-53228 and those described in U.S. Pat. No. 5,451,700 and WO 04/108720.

Leukotrienes are products derived from arachidonic acid that act on smooth muscles and can be responsible for respiratory and inflammatory diseases such as asthma and arthritis. Leukotriene D4 antagonists inhibit the LTD4 receptor. Such compounds are useful in the treatment of conditions which respond to the inhibition of the LTD4 receptor, particularly inflammatory or allergic conditions. Suitable LTD4 antagonists include montelukast, pranlukast, zafirlukast, accolate, SR2640, Wy-48,252, ICI 198615, MK-571, LY-171883, Ro 24-5913 and L-648051.

A serine protease inhibitor is a substance or agent that inhibits a serine protease. Serine proteases include trypsin, matriptase, prostasin (PRSS8), plasmin, tPA, uPA, Xa, IXa, thrombin, tissue factor, compliment factors, tryptase, HNE, kallikrein (plasma and tissue), matriptase and TRMPSS 3 and 4. Serine protease inhibitors also include channel activating protease inhibitors such as antipain, aprotinin, benzamidine, camostat, gabexate, leupeptin, nafamostat, pepstatin A, ribavirin, sepimostat and ulinastatin. Suitable trypsin inhibitors include patamostat mesylate and those compounds generally or specifically described in U.S. Pat. No. 6,469,036, e.g. RWJ-58643 (J&J), EP 556024, e.g. TO-195 (Torii), U.S. Pat. No. 6,469,036, e.g. RWJ-56423 (Ortho-McNeil), JP96020570, e.g. TT-S24 (Teikoko Chemical), EP588655 and WO0181314. Matriptase and prostasin (PRSS8) inhibitors are known as trypsin-like serine protease inhibitors. Suitable Xa inhibitors include fondaparin sodium, rivaroxaban, idrapainux sodium, apixaban and otamixaban and those compounds specifically and generally described in U.S. Pat. No. 6,469,036, particularly RWJ-58643 (J&J), U.S. Pat. No. 6,022,861, U.S. Pat. No. 6,211,154, particularly MLN-1021 (Millennium), FR2773804, e.g. SR123781 (Sanofi-Aventis), DE 19829964, e.g. tanogitran, U.S. Pat. No. 6,469,026, WO 00/01704, e.g. BIBR-1109 (Boehringer Ingelheim), DE 19829964, e.g. BIBT-0871, BIBT-1011 and BIBT-0932CL (Boehringer Ingelheim) and DE19816983. Other Factor Xa inhibitors for use in the present invention include those compounds specifically disclosed in the review document Expert Opin. Ther. Patents (2006) 16(2):119-145, e.g. DX-9065a, DPC-423, Razaxaban, BAY59-7938 and compounds number 5-153. Suitable thrombin inhibitors include argatroban, glycyrrhizin (Ligand), odiparcil, corthrombin, those compounds specifically and generally described in U.S. Pat. No. 5,523,308 (J&J), WO 91/02750, e.g. Hirulog-1 (Biogen), DE 19706229, e.g. dabigratan and dabigratan etexilate, AU 8551553, e.g. efegatran sulfate hydrate, WO 93/11152, e.g. inogatran, US 2003134801, e.g. LB-30870 (LG Chem), Org42675 (Akzo Nobel), EP 559046, e.g. napsagatran, WO 01/070736, e.g. SSR-182289, EP 615978, e.g. S-18326 (Servier), WO 95/13274, e.g. UK-156406 (Pfizer), EP 0918768, e.g. AT-1362 (C&C Research Labs), WO 00/55156, e.g. AT-1459 (C&C Research Labs), JP 1999502203, e.g. BCH-2763 (Nat Res Council of Canada), EP623596, e.g. BMS-189090 (BMS), CA 2151412, e.g. BMS-191032 (BMS), U.S. Pat. No. 5,037,819, e.g. BMY-43392-1 (BMS), GB 2312674, e.g. CGH-1484A (Novartis), EP 739886, e.g. CI-1028, LB-30057 and PD-172524 (LG Chem), DE 4115468, e.g. CRC-220 (Dade Behring Marburg), AU 8817332, e.g. DuP-714 (BMS), JP 96333287, e.g. F-1070 (Fuji Yakuhin), WO 97/01338, e.g. L-373890, L-374087 and L-375052 (Merck), WO 97/40024, e.g. L-375378 (Merck), WO 98/42342, e.g. L-376062 (Merck), WO 02/51824, e.g. LK-658 and LK-732 (Lek), WO 97/05160, e.g. LR-D/009 (Guidotti), EP 479489, e.g. LY-293435 (Lilly), AU 8945880, e.g. MDL-28050 (Sanofi Avenits), EP 195212, e.g. MDL-73756 (Sanofi Avenits), AU 9059742, e.g. MDL-74063 (Sanofi Avenits), JP 90289598, e.g. Cyclotheonamide A, WO 99/65934, e.g. NAPAP-PS (Organon), E0858464, e.g. Org-37432 (Organon), WO 98/47876, e.g. Org-37476 (Organon), WO 98/07308, e.g. Org-39430 (Organon), EP 217286, e.g. OS-396, CA 2152205, e.g. S-30266 (Adir), EP 792883, e.g. S-31214 and S-31922 (Servier), EP 471651, e.g. SDZ-217766 and SDZ-MTH-958 (Novartis), WO 95/13274, e.g. UK-179094 (Pfizer), WO 97/16444, e.g. UK-285954 (Pfizer), WO 98/01428, e.g. XU-817 (BMS), JP 96020597, U.S. Pat. No. 5,510,369, WO 97/36580, WO 98/47876, WO98/47876, WO 97/46553, WO 98/42342, WO 97/46553, EP 863755, U.S. Pat. No. 5,891,909, WO 99/15169, EP 0815103, US 6117888, WO 00/75134, WO 00/75134, WO 01/38323, EP 00944590, WO 02/64140, EP 1117660, EP 0944590 and EP 0944590. Suitable tryptase inhibitors include mast cell tryptase inhibitors such as those compounds specifically and generally described in WO 94/20527, particularly APC-366 (Celera), and the compounds APC-2059 (Bayer), AVE-8923 (Sanofi-Aventis), MOL-6131 (Molecumetics) and M-58539 (Mochida). Suitable kallikrein inhibitors include cetraxate and ecallanitide.

Suitable PDE4 inhibitors PDE4 inhibitors such as cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SelCID™ CC-10004 (Celgene), VM554/UM565 (Vernalis), T-440 (Tanabe), KW-4490 (Kyowa Hakko Kogyo), GRC 3886 (Oglemilast, Glenmark), GSK256066, and those described in WO 92/19594, WO 93/19749, WO 93/19750, WO 93/19751, WO 98/18796, WO 99/16766, WO 01/13953, WO 03/39544, WO 03/104204, WO 03/104205, WO 04/000814, WO 04/000839, WO 04/005258, WO 04018450, WO 04/018451, WO 04/018457, WO 04/018465, WO 04/018431, WO 04/018449, WO 04/018450, WO 04/018451, WO 04/018457, WO 04/018465, WO 04/019944, WO 04/019945, WO 04/045607, WO 04/037805, WO 04/063197, WO 04/103998, WO 04/111044, WO 05012252, WO 05012253, WO 05/013995, WO 05/030725, WO 05/030212, WO 05/087744, WO 05/087745, WO 05/087749 and WO 05/090345.

Compounds of the invention (i.e. compounds of A and/or B) that contain a basic centre are capable of forming acid addition salts, particularly pharmaceutically acceptable acid addition salts. Pharmaceutically acceptable acid addition salts of the compounds of the invention include those of inorganic acids, for example, hydrohalic acids such as hydrofluoric acid, hydrochloric acid, hydrobromic acid, hydroiodic acid, nitric acid, sulfuric acid, phosphoric acid; and organic acids, for example aliphatic monocarboxylic acids such as formic acid, acetic acid, trifluoroacetic acid, propionic acid and butyric acid, caprylic acid, dichloroacetic acid, hippuric acid, aliphatic hydroxy acids such as lactic acid, citric acid, tartaric acid or malic acid, gluconic acid, mandelic acid, dicarboxylic acids such as maleic acid or succinic acid, adipic acid, aspartic acid, fumaric acid, glutamic acid, malonic acid, sebacic acid, aromatic carboxylic acids such as benzoic acid, p-chloro-benzoic acid, nicotinic acid, diphenylacetic acid or triphenylacetic acid, aromatic hydroxy acids such as o-hydroxybenzoic acid, p-hydroxybenzoic acid, 1-hydroxynaphthalene-2-carboxylic acid or 3-hydroxynaphthalene-2-carboxylic acid, and sulfonic acids such as methanesulfonic acid or benzenesulfonic acid, ethanesulfonic acid, ethane-1,2-disulfonic acid, 2-hydroxy-ethanesulfonic acid, (+) camphor-10-sulfonic acid, naphthalene-2-sulfonic acid, naphthalene-1,5-disulfonic acid or p-toluenesulfonic acid. These salts may be prepared from compounds of the invention by known salt-forming procedures. Pharmaceutically acceptable solvates are generally hydrates.

Compounds of the invention which contain acidic, e.g. carboxyl, groups, are also capable of forming salts with bases, in particular pharmaceutically acceptable bases such as those well known in the art; suitable such salts include metal salts, particularly alkali metal or alkaline earth metal salts such as sodium, potassium, magnesium or calcium salts, or salts with ammonia or pharmaceutically acceptable organic amines or heterocyclic bases such as ethanolamines, benzylamines or pyridine, arginine, benethamine, benzathine, diethanolamine, 4-(2-hydroxy-ethyl)morpholine, 1-(2-hydroxyethyl)pyrrolidine, N-methyl glutamine, piperazine, triethanol-amine or tromethamine. These salts may be prepared from compounds of the invention by known salt-forming procedures. Compounds of the invention that contain acidic, e.g. carboxyl, groups may also exist as zwitterions with the quaternary ammonium centre.

Compounds of the invention in free form may be converted into salt form, and vice versa, in a conventional manner. The compounds in free or salt form can be obtained in the form of hydrates or solvates containing a solvent used for crystallisation. Compounds of the invention can be recovered from reaction mixtures and purified in a conventional manner. Isomers, such as enantiomers, may be obtained in a conventional manner, e.g. by fractional crystallisation or asymmetric synthesis from correspondingly asymmetrically substituted, e.g. optically active, starting materials.

Some compounds of the invention contain at least one asymmetric carbon atom and thus they exist in individual optically active isomeric forms or as mixtures thereof, e.g. as racemic mixtures. In cases where additional asymmetric centres exist the present invention also embraces both individual optically active isomers as well as mixtures, e.g. diastereomeric mixtures, thereof.

The invention includes all such forms, in particular the pure isomeric forms. The different isomeric forms may be separated or resolved one from the other by conventional methods, or any given isomer may be obtained by conventional synthetic methods or; by stereospecific or asymmetric syntheses. Since the compounds of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the invention.

The invention includes all pharmaceutically acceptable isotopically-labelled compounds of the invention wherein one or more atoms are replaced by atoms having the same atomic number, but an atomic mass or mass number different from the atomic mass or mass number usually found in nature. Examples of isotopes suitable for inclusion in the compounds of the invention include isotopes of hydrogen e.g. ²H and ³H, carbon e.g. ¹¹C, ¹³C and ¹⁴C, chlorine e.g. ³⁶Cl, fluorine e.g. ¹⁸F, iodine e.g. ¹²³I and ¹²⁵I, nitrogen e.g. ¹³N and ¹⁵N, oxygen e.g. ¹⁵O, ¹⁷O and ¹⁸O, and sulfur e.g. ³⁵S.

Certain isotopically-labelled compounds of the invention, for example those incorporating a radioactive isotope, are useful in drug and/or substrate tissue distribution studies. The radioactive isotopes tritium (³H) and carbon-14 (¹⁴C) are particularly useful for this purpose in view of their ease of incorporation and ready means of detection. Substitution with heavier isotopes such as deuterium (²H) may afford certain therapeutic advantages that result from greater metabolic stability, for example increased in vivo half-life or reduced dosage requirements, and hence may be preferred in some circumstances. Substitution with positron emitting isotopes, such as ¹¹C, ¹⁸F, ¹⁵O, and ¹³N can be useful in Positron Emission Topography (PET) studies for examining substrate receptor occupancy.

Isotopically-labelled compounds of the invention can generally be prepared by conventional techniques known to those skilled in the art or by processes analogous to those described in the accompanying examples using an appropriate isotopically-labelled reagent in place of the non-labelled reagent previously used.

Pharmaceutically acceptable solvates in accordance with the invention include those wherein the solvent of crystallisation may be isotopically substituted e.g. D₂O, d₆-acetone or d₆-DMSO.

The compounds of (A) activate the adenosine Am receptor, i.e. they act as A_{2A} receptor agonists. Their properties as A_{2A} agonists may be demonstrated using the method described by L. J. Murphree et al in Molecular Pharmacology 61, 455-462 (2002).

Compounds of the Examples hereinbelow have Kᵢ values below 1.0 μM in the above assay. For example, the compound of Example 1 has a Ki value of 0.004 μM.

Having regard to the fact that the compounds of (A) activate the adenosine A_{2A} receptor, the medicament or pharmaceutical composition according to the invention, hereinafter alternately referred to as “agents of the invention”, are useful in the treatment of conditions which respond, at least in part, to the activation of the adenosine A_{2A} receptor, particularly inflammatory or allergic conditions. Treatment in accordance with the invention may be symptomatic or prophylactic.

Accordingly, agents of the invention are useful in the treatment of inflammatory or obstructive airways diseases, resulting, for example, in reduction of tissue damage, airways inflammation, bronchial hyperreactivity, remodelling or disease progression.

Administration of the medicament or pharmaceutical composition as hereinbefore described, i.e. with (A) and (B) in admixture or separate, is suitably by inhalation, i.e. (A) and (B) or the mixture thereof are in inhalable form. The inhalable form of the medicament i.e. of (A) and/or (B) may be, for example, an atomizable composition such as an aerosol comprising the active ingredient, i.e. (A) and (B) separately or in admixture, in solution or dispersion in a propellant, or a nebulizable composition comprising a solution or dispersion of the active ingredient in an aqueous, organic or aqueous/organic medium. For example, the inhalable form of the medicament may be an aerosol comprising a mixture of (A) and (B) in solution or dispersion in a propellant, or a combination of an aerosol containing (A) in solution or dispersion in a propellant with an aerosol containing (B) in solution or dispersion in a propellant. In another example, the inhalable form is a nebulizable composition comprising a dispersion of (A) and (B) in an aqueous, organic or aqueous/organic medium, or a combination of a dispersion of (A) in such a medium with a dispersion of (B) in such a mediums.

An aerosol composition suitable for use as the inhalable form of the medicament may comprise the active ingredient in solution or dispersion in a propellant, which may be chosen from any of the propellants known in the art. Suitable such propellants include hydrocarbons such as n-propane, n-butane or isobutane or mixtures of two or more such hydrocarbons, and halogen-substituted hydrocarbons, for example chlorine and/or fluorine-substituted methanes, ethanes, propanes, butanes, cyclopropanes or cyclobutanes, such as dichlorodifluoromethane (CFC 12), trichlorofluoromethane (CFC11), 1,2-dichloro-1,1,2,2-tetrafluoroethane (CFC114) or, particularly, 1,1,1,2-tetrafluoroethane (HFA134a) and 1,1,1,2,3,3,3-heptafluoropropane (HFA227), or mixtures of two or more such halogen-substituted hydrocarbons. Where the active ingredient is present in suspension in the propellant, i.e. where it is present in particulate form dispersed in the propellant, the aerosol composition may also contain a lubricant and a surfactant, which may be chosen from those lubricants and surfactants known in the art. Other suitable aerosol compositions include surfactant-free or substantially surfactant-free aerosol compositions. The aerosol composition may contain up to about 5% by weight, for example 0.0001 to 5%, 0.001 to 5%, 0.001 to 3%, 0.001 to 2%, 0.001 to 1%, 0.001 to 0.1%, or 0.001 to 0.01% by weight of the active ingredient, based on the weight of the propellant. Where present, the lubricant and surfactant may be in an amount up to 5% and 0.5% respectively by weight of the aerosol composition. The aerosol composition may also contain a co-solvent such as ethanol in an amount up to 30% by weight of the composition, particularly for administration from a pressurised metered dose inhalation device. The aerosol composition may further contain a bulking agent, for example a sugar such as lactose, sucrose, dextrose, mannitol or sorbitol, in an amount, for example, of up to 20%, usually 0.001 to 1%, by weight of the composition.

In another embodiment of the invention, the inhalable form is a dry powder, i.e. (A) and/or (B) are present in a dry powder comprising finely divided (A) and/or (B) optionally together with at least one particulate pharmaceutically acceptable carrier, which may be one or more materials known as pharmaceutically acceptable carriers, optionally chosen from materials known as carriers in dr powder inhalation compositions, for example saccharides, including monosaccharides, disaccharides, polysaccharides and sugar alcohols such as arabinose, glucose, fructose, ribose, mannose, sucrose, trehalose, lactose, maltose, starches, dextran, mannitol or sorbitol. An especially suitable carrier is lactose. The dr powder may be contained as unit doses in capsules of, for example, gelatin or plastic, or in blisters (e.g. of aluminium or plastic), for use in a dr powder inhalation device, which may be a single dose or multiple dose device, suitably in dosage units of (A) and/or (B) together with the carrier in amounts to bring the total weight of powder per capsule to from 5 mg to 50 mg. Alternatively, the dr powder may be contained in a reservoir in a multi-dose dry powder inhalation device adapted to deliver, for example, 3-25 mg of dr powder per actuation.

In the finely divided particulate form of the medicament, and in the aerosol composition where the active ingredient is present in particulate form, the active ingredient may have an average particle diameter of up to about 10 μm, for example 0.1 to 5 μm, preferably 1 to 5 μm. The particulate carrier, where present, generally has a maximum particle diameter up to 300 μm, preferably up to 212 μm, and conveniently has a mean particle diameter of 40 to 100 μm, e.g. 50 to 75 μm. The particle size of the active ingredient, and that of a particulate carrier where present in dry powder compositions, can be reduced to the desired level by conventional methods, for example by grinding in an air-jet mill, ball mill or vibrator mill, sieving, microprecipitation, spray-drying, lyophilisation or controlled crystallisation from conventional solvents or from supercritical media.

The inhalable medicament may be administered using an inhalation device suitable for the inhalable form, such devices being well known in the art. Accordingly, the invention also provides a pharmaceutical product comprising a medicament or pharmaceutical composition as hereinbefore described in inhalable form as hereinbefore described in association with one or more inhalation devices. In a further aspect, the invention provides an inhalation device, or a pack of two or more inhalation devices, containing a medicament or pharmaceutical composition as hereinbefore described in inhalable form as hereinbefore described.

Where the inhalable form of the active ingredient is an aerosol composition, the inhalation device may be an aerosol vial provided with a valve adapted to deliver a metered dose, such as 10 to 100 μl, e.g. 25 to 50 μl, of the composition, i.e. a device known as a metered dose inhaler. Suitable such aerosol vials and procedures for containing within them aerosol compositions under pressure are well known to those skilled in the art of inhalation therapy. For example, an aerosol composition may be administered from a coated can, for example as described in EP-A-0642992. Where the inhalable form of the active ingredient is a nebulizable aqueous, organic or aqueous/organic dispersion, the inhalation device may be a known nebulizer, for example a conventional pneumatic nebulizer such as an airjet nebulizer, or an ultrasonic nebulizer, which may contain, for example, from 1 to 50 ml, commonly 1 to 10 ml, of the dispersion; or a hand-held nebulizer, sometimes referred to as a soft mist or soft spray inhaler, for example an electronically controlled device such as an AERx (Aradigm, US) or Aerodose (Aerogen), or a mechanical device such as a RESPIMAT (Boehringer Ingelheim) nebulizer which allows much smaller nebulized volumes, e.g. 10 to 100 μl, than conventional nebulizers. Where the inhalable form of the active ingredient is the finely divided particulate form, the inhalation device may be, for example, a dr powder inhalation device adapted to deliver dr powder from a capsule or blister containing a dry powder comprising a dosage unit of (A) and/or (B) or a multidose dr powder inhalation (MDPI) device adapted to deliver, for example, 3-25 mg of dr powder comprising a dosage unit of (A) and/or (B) per actuation. The dry powder composition suitably contains a diluent or carrier, such as lactose, and a compound that helps to protect against product performance deterioration due to moisture e.g. magnesium stearate. Suitable such dr powder inhalation devices are well known. For example, a suitable device for delivery of dry powder in encapsulated form is that described in U.S. Pat. No. 3,991,761, while a suitable MDPI device is that described in WO 97/20589.

The medicament of the invention is suitably a pharmaceutical composition comprising a mixture of (A) as hereinbefore defined and (B) as hereinbefore defined, optionally together with at least one pharmaceutically acceptable carrier as hereinbefore described.

The molar ratio of the compound (A) to the steroid (B) may be, in general, from 100:1 to 1:300, for example from 50:1 to 1:100 or from 20:1 to 1:50, preferably from 10:1 to 1:20, more preferably from 5:1 to 1:10, from 3:1 to 1:7 or from 2:1 to 1:2. The component (A) and the component (B) may be administered separately in the same ratio.

A suitable daily dose of (A) for inhalation may be from 10 μg to 5000 μg, for example from 20 to 4000 μg, from 50 to 3000 μg, from 50 to 2000 μg, from 50 to 1000 μg, from 50 to 500 μg, from 50 to 400 μg, from 50 to 300 μg, from 50 to 200 μg or from 50 to 100 μg.

The molar ratio of the compound (A) to the LABA (B) may be, in general, from 300:1 to 1:300, for example from 100:1 to 1:100 or from 50:1 to 1:50, preferably from 20:1 to 1:20, more preferably from 10:1 to 1:10, from 5:1 to 1:5 or from 2:1 to 1:2. The component (A) and the component (B) may be administered separately in the same ratio.

The molar ratio of the compound (A) to the LAMA (B) may be, in general, from 300:1 to 1:300, for example from 100:1 to 1:100 or from 50:1 to 1:50, preferably from 20:1 to 1:20, more preferably from 10:1 to 1:10, from 5:1 to 1:5 or from 2:1 to 1:2. The component (A) and the component (B) may be administered separately in the sane ratio.

Where (B) is an A_{2B} antagonists, a suitable daily dose for inhalation may be from 20 μg to 5000 μg, for example from 20 to 4000 μg, from 50 to 3000 μg, from 50 to 2000 μg, from 50 to 1000 μg, from 50 to 500 μg, from 50 to 400 μg, from 50 to 300 μg, from 50 to 200 μg or from 50 to 100 μg.

Where (B) is an antihistamine, a suitable daily dose for inhalation may be from 20 μg to 5000 μg, for example from 20 to 4000 μg, from 50 to 3000 μg, from 50 to 2000 μg, from 50 to 1000 μg, from 50 to 500 μg, from 50 to 400 μg, from 50 to 300 μg, from 50 to 200 μg or from 50 to 100 μg.

Where (B) is a caspase inhibitor, a suitable daily dose for inhalation may be from 20 μg to 5000 μg, for example from 20 to 4000 μg, from 50 to 3000 μg, from 50 to 2000 μg, from 50 to 1000 μg, from 50 to 500 μg, from 50 to 400 μg, from 50 to 300 μg, from 50 to 200 μg or from 50 to 100 μg.

Where (B) is an ENaC inhibitor, a suitable daily dose for inhalation may be from 20 μg to 5000 μg, for example from 20 to 4000 μg, from 50 to 3000 μg, from 50 to 2000 μg, from 50 to 1000 μg, from 50 to 500 μg, from 50 to 400 μg, from 50 to 300 μg, from 50 to 200 μg or from 50 to 100 μg.

Where (B) is a LTB4 antagonist, a suitable daily dose for inhalation may be from 20 μg to 5000 μg, for example from 20 to 4000 μg, from 50 to 3000 μg, from 50 to 2000 μg, from 50 to 1000 μg, from 50 to 500 μg, from 50 to 400 μg, from 50 to 300 μg, from 50 to 200 μg or from 50 to 100 μg.

Where (B) is a LTD4 antagonist, a suitable daily dose for inhalation may be from 20 μg to 5000 μg, for example from 20 to 4000 μg, from 50 to 3000 μg, from 50 to 2000 μg, from 50 to 1000 μg, from 50 to 500 μg, from 50 to 400 μg, from 50 to 300 μg, from 50 to 200 μg or from 50 to 100 μg.

Where (B) is a serine protease inhibitor, a suitable daily dose for inhalation may be from 20 μg to 5000 μg, for example from 20 to 4000 μg, from 50 to 3000 μg, from 50 to 2000 μg, from 50 to 1000 μg, from 50 to 500 μg, from 50 to 400 μg, from 50 to 300 μg, from 50 to 200 μg or from 50 to 100 μg.

In an embodiment of the invention, the medicament of the invention is a pharmaceutical composition which is a dry powder in a capsule containing a unit dose of (A) and (B), for example for inhalation from a single capsule inhaler, the capsule suitably containing a unit dose of (A) e.g. as hereinbefore described, and a unit dose of (B), e.g. as hereinbefore described, together with a pharmaceutically acceptable carrier as hereinbefore described in an amount to bring the total weight of dry powder per capsule to between 5 mg and 50 mg, for example 5 mg, 10 mg, 15 mg, 20 mg, 25 mg, 30 mg, 35 mg, 40 mg, 45 mg or 50 mg.

In another embodiment of the invention, the medicament of the invention is a pharmaceutical composition which is a dr powder for administration from a reservoir of a multi-dose dr powder inhaler adapted to deliver, for example, 3 mg to 25 mg of powder containing a unit dose of (A) and (B) per actuation, for example, where (A) is in the form of a salt, a powder comprising, by weight, 20 to 2000 parts, for example 60 to 1000 parts, 100 to 500 parts, or 100 to 300 parts of (A); 25 to 800 parts, e.g. 25 to 500 parts, 50 to 400 parts, or 100 to 400 parts of (B); and 2000 to 25000 parts, e.g. 4000 to 15000 parts or 4000 to 10000 parts of a pharmaceutically acceptable carrier as hereinbefore described.

In a further embodiment of the invention, the medicament of the invention is a pharmaceutical composition which is an aerosol comprising (A) and (B), e.g. in a ratio as hereinbefore described, in a propellant as hereinbefore described, optionally together with a surfactant and/or a bulking agent and/or a co-solvent such as ethanol as hereinbefore described, for administration from a metered dose inhaler adapted to deliver an amount of aerosol containing a unit dose of (A) and a unit dose of (B), or a known fraction of a unit dose of (A) and a known fraction of a unit dose of (B), per actuation. Thus if, for example, the inhaler delivers half of the unit doses of (A) and (B) per actuation, the unit doses can be administered by two actuations of the inhaler.

In accordance with the above, the invention also provides a pharmaceutical kit comprising (A) and (B) as hereinbefore defined in separate unit dosage forms, said forms being suitable for administration of (A) and (B) in effective amounts. Such a kit suitably further comprises one or more inhalation devices for administration of (A) and (B). For example, the kit may comprise one or more dr powder inhalation devices adapted to deliver dry powder from a capsule, together with capsules containing a dry powder comprising a dosage unit of (A) and capsules containing a dry powder comprising a dosage unit of (B). In another example, the kit may comprise a multidose dry powder inhalation device containing in the reservoir thereof a dry powder comprising (A) and a multidose dry powder inhalation device containing in the reservoir thereof a dry powder comprising (B). In a further example, the kit may comprise a metered dose inhaler containing an aerosol comprising (A) in a propellant and a metered dose inhaler containing an aerosol comprising (B) in a propellant.

The medicaments of the invention are advantageous in the treatment of inflammatory or obstructive airways disease, exhibiting highly effective bronchodilatory and anti-inflammatory properties. For instance, it is possible using the combination therapy of the invention to reduce the dosages of corticosteroid required for a given therapeutic effect compared with those required using treatment with a corticosteroid alone, thereby minimising possibly undesirable side effects. In particular, these combinations, particularly where (A) and (B) are in the same composition, facilitate achievement of a high anti-inflammatory effect, such that the amount of corticosteroid needed for a given anti-inflammatory effect may be reduced it forms at least one component of (B) or when used in admixture with the present invention, thereby reducing the risk of undesirable side effects from the repeated exposure to the steroid involved in the treatment of inflammatory or obstructive airways diseases. Furthermore, using the combinations of the invention, particularly using compositions containing (A) and (B), medicaments which have a rapid onset of action and a long duration of action may be prepared. Moreover, using such combination therapy, medicaments which result in a significant improvement in lung function may be prepared. In another aspect, using the combination therapy of the invention, medicaments which provide effective control of obstructive or inflammatory airways diseases, or a reduction in exacerbations of such diseases, may be prepared. In a further aspect, using compositions of the invention containing (A) and (B), medicaments which reduce or eliminate the need for treatment with short-acting rescue medicaments such as salbutamol or terbutaline, may be prepared; thus compositions of the invention containing (A) and (B) facilitate the treatment of an obstructive or inflammatory airways disease with a single medicament.

Treatment of inflammatory or obstructive airways diseases in accordance with the invention may be symptomatic or prophylactic treatments Inflammatory or obstructive airways diseases to which the present invention is applicable include chronic obstructive pulmonary, airways or lung disease (COPD, COAD or COLD), including chronic bronchitis and emphysema, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection. Treatment of asthma is also to be understood as embracing treatment of subjects, e.g. of less than 4 or 5 years of age, exhibiting wheezing symptoms and diagnosed or diagnosable as “wheezy infants”, an established patient category of major medical concern and now often identified as incipient or early-phase asthmatics. (For convenience this particular asthmatic condition is referred to as “wheezy-infant syndrome”.)

Prophylactic efficacy in the treatment of asthma will be evidenced by reduced frequency or severity of symptomatic attack, e.g. of acute asthmatic or bronchoconstrictor attack, improvement in lung function or improved airways hyperreactivity. It may further be evidenced by reduced requirement for other, symptomatic therapy, i.e. therapy for or intended to restrict or abort symptomatic attack when it occurs, for example anti-inflammatory (e.g. corticosteroid) or bronchodilatory. Prophylactic benefit in asthma may in particular be apparent in subjects prone to “morning dipping”. “Morning dipping” is a recognised asthmatic syndrome, common to a substantial percentage of asthmatics and characterised by asthma attack, e.g. between the hours of about 4 to 6 am, i.e. at a time normally substantially distant form any previously administered symptomatic asthma therapy.

Other inflammatory or obstructive airways diseases and conditions to which the present invention is applicable include acute lung injury (ALI), adult respiratory distress syndrome (ARDS), cystic fibrosis, bronchiectasis and exacerbation of airways hyperreactivity consequent to other drug therapy, in particular other inhaled drug therapy. Further inflammatory or obstructive airways diseases to which the present invention is applicable include pneumoconiosis (an inflammatory, commonly occupational, disease of the lungs, frequently accompanied by airways obstruction, whether chronic or acute, and occasioned by repeated inhalation of dusts) of whatever type or genesis, including, for example, pulmonary fibrosis, aluminosis, anthracosis, asbestosis, chalicosis, ptilosis, siderosis, silicosis, tobacosis and byssinosis.

Synthetic methods for preparing the compounds of (A) are described in the Examples section below.

### EXAMPLES

TABLE 1

| Ex. | Structure | [M + H]⁺ |
|---|---|---|
| 1 | 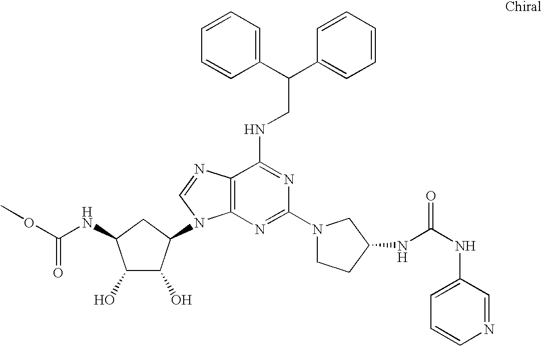 | 693 |
| 1a | 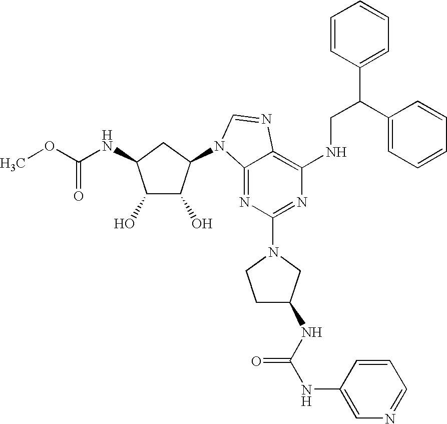 | 693 |
| 2 | 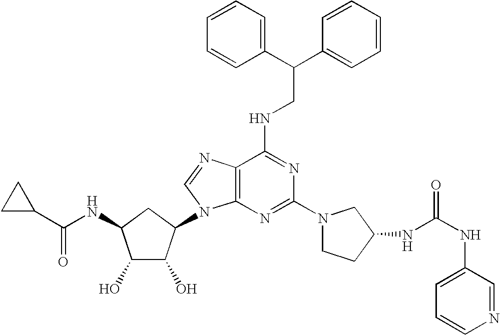 | 704 |
| 3 | 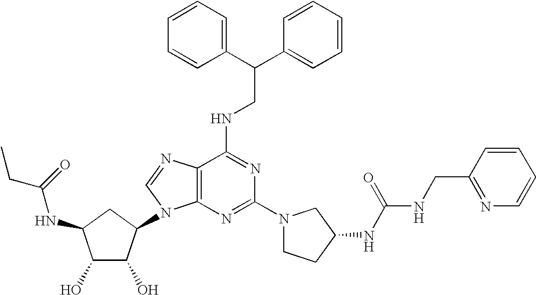 | 705 |
| 4 | 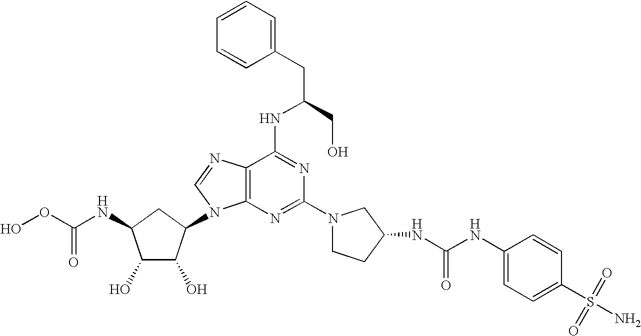 | 705 |
| 5 | 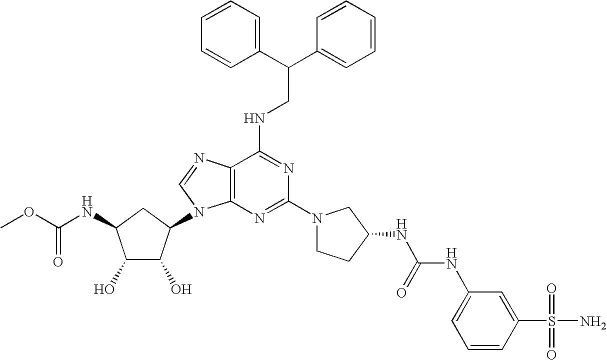 | 771 |
| 6 | 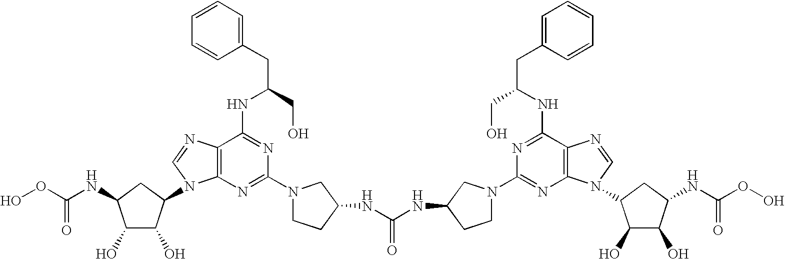 | 540 [M + 2H]²⁺ |
| 7 | 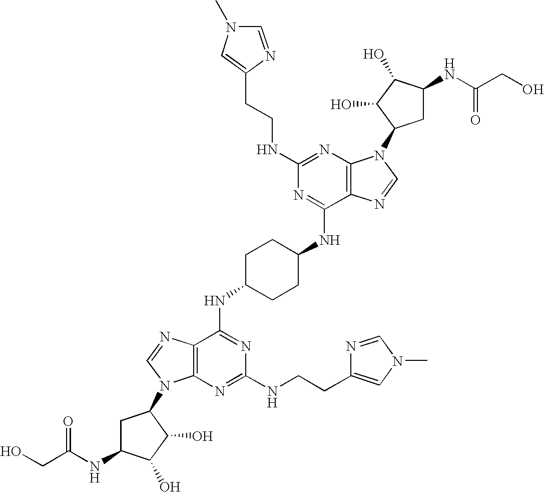 | 472 [M + 2H]²⁺ |

In the Experimental Section the following abbreviations have been used:

RT room temperature

DMF dimethyl-formamide

DIPEA diisopropylethylamine

NMP N-methylpyrrolidine

THF tetrahydrofuran

MeOH methanol

DCM dichloromethane

EtOAc ethyl acetate

EtOH ethanol

LCMS liquid chromatographic mass spectroscopy

TEA triethylamine

TFA trifluoroacetic acid

HPLC High Performance Liquid Chromatography

HCl Hydrochloric Acid

CDI carbonyl diimidazole

The following standard chemical reagents within the common general knowledge of the skilled chemist have been utilized: Hunig's base. Methods of preparation of such compounds are well-known.

In addition various trade reagents and materials available from have been utilized. Such reagents and materials include: Isolute™ (available from Biotage) and can be readily obtained from the suppliers indicated.

Compounds are identified using either: commonly used non-systematic nomenclature, or generated using AutoNom software.

Mass spectra are run on LCMS systems using electrospray ionization. These are either Agilent 1100 HPLC/Micromass Platform Mass Spectrometer combinations or Waters Acquity HPLC with SQD Mass Spectrometer. [M+H]⁺ refers to mono-isotopic molecular weights.

NMR spectra are run on Bruker AVANCE 400 NMR spectrometers using ICON-NMR. Spectra are measured at 298K and are referenced using the solvent peak.

### Example 1

### ((1S,2R,3S,4R)-4-{6-(2,2-Diphenyl-ethylamino)-2-[(R)-3-(3-pyridin-3-yl-ureido)-pyrrolidin-1-yl]-purin-9-yl}-2,3-dihydroxy-cyclopentyl)-carbamic acid methyl ester hydrochloride

Step 1: ((1S,2R,3S,4R)-4-{6-(2,2-Diphenyl-ethylamino)-2-[(R)-3-(3-pyridin-3-yl-ureido)-pyrrolidin-1-yl]-purin-9-yl}-2,3-dihydroxy-cyclopentyl)-carbamic acid benzyl ester trifluoroacetate

A solution comprising {(1S,2R,3S,4R)-4-[2-((R)-3-amino-pyrrolidin-1-yl)-6-(2,2-diphenyl-ethylamino)-purin-9-yl]-2,3-dihydroxy-cyclopentyl}-carbamic acid benzyl ester (Intermediate C) (0.1 g, 0.15 mmol), pyridine-3-isocyanate (0.02 g, 0.17 mmol) and TEA (0.017 g, 0.17 mmol) in THF (2 ml) is stirred at room temperature overnight. The solvent is removed in vacuo and purification is carried out by reverse phase column chromatography (Isolute™ C18, 0-100% acetonitrile in water—0.1% TFA). The fractions are collected and the MeCN is removed in vacuo. The remaining aqueous portion is basified with saturated sodium bicarbonate solution and extracted with DCM. The combined organic extracted are dried (MgSO₄) and concentrated in vacuo to afford the title product. [M+H]⁺ 769.

Step 2: 1-{(R)-1-[9-((1R,2S,3R,4S)-4-Amino-2,3-dihydroxy-cyclopentyl)-6-(2,2-diphenyl-ethylamino)-9H-purin-2-yl]-pyrrolidin-3-yl}-3-pyridin-3-yl-urea

To a solution of ((1S,2R,3S,4R)-4-{6-(2,2-diphenyl-ethylamino)-2-[(R)-3-(3-pyridin-3-yl-ureido)-pyrrolidin-1-yl]-purin-9-yl}-2,3-dihydroxy-cyclopentyl)-carbamic acid benzyl ester trifluoroacetate (step 1) (35 mg, 46 μmol) in ethanol (1 ml) under an atmosphere of argon is added 10% palladium on carbon (10 mg). The reaction mixture is purged with argon and placed under an atmosphere of hydrogen overnight after which time, the mixture is filtered through celite® (filter material) and the catalyst washed with ethanol. The organic portions are combined and concentrated in vacuo to yield the title compound. [M+H]⁺ 635

Step 3: ((1S,2R,3S,4R)-4-{6-(2,2-Diphenyl-ethylamino)-2-[(R)-3-(3-pyridin-3-yl-ureido)-pyrrolidin-1-yl]-purin-9-yl}-2,3-dihydroxy-cyclopentyl)-carbamic acid methyl ester hydrochloride

A solution of 1-{(R)-1-[9-((1R,2S,3R,4S)-4-amino-2,3-dihydroxy-cyclopentyl)-6-(2,2-diphenyl-ethylamino)-9H-purin-2-yl]-pyrrolidin-3-yl}-3-pyridin-3-yl-urea (11 mg, 17 μmol) in THF (0.5 ml) at room temperature is treated with DIPEA (7 mg) followed by methyl chloroformate (1.8 mg) added as a 10% solution in THF. The reaction mixture becomes cloudy and NMP (0.1 nm) is added to aid dissolution. The resulting reaction mixture is stirred at room temperature for 30 minutes and then the solvent in vacuo. The crude solid is dissolved in MeOH (1 ml), treated with NaHCO₃ (5 equivalents) and left at room temperature overnight. Purification of the resulting mixture by reverse phase column chromatography (Isolute™ C18, 0-100% acetonitrile in water—0.1% HCl) affords the title compound. [M+H]⁺ 693.

### Example 1a

### ((1S,2R,3S,4R)-4-{6-(2,2-Diphenyl-ethylamino)-2-[(S)-3-(3-pyridin-3-yl-ureido)-pyrrolidin-1-yl]-purin-9-yl}-2,3-dihydroxy-cyclopentyl)-carbamic acid methyl ester

The title compound is prepared analogously to Example 1 by replacing {(1S,2R,3S,4R)-4-[2-((R)-3-amino-pyrrolidin-1-yl)-6-(2,2-diphenyl-ethylamino)-purin-9-yl]-2,3-dihydroxy-cyclopentyl}-carbamic acid benzyl ester (Intermediate C) with the appropriate isomeric starting material.

### Example 2

### Cyclopropanecarboxylic acid ((1S,2R,3S,4R)-4-{6-(2,2-diphenyl-ethylamino)-2-[(R)-3-(3-pyridin-3-yl-ureido)-pyrrolidin-1-yl]-purin-9-yl}-2,3-dihydroxy-cyclopentyl)-amide

This compound is prepared analogously to Example 1 by replacing methyl chloroformate with cyclopropanecarbonyl chloride. [M+H]⁺ 704.

### Example 3

### N-((1S,2R,3S,4R)-4-{6-(2,2-Diphenyl-ethylamino)-2-[(R)-3-(3-pyridin-2-ylmethyl-ureido)-pyrrolidin-1-yl]-purin-9-yl}-2,3-dihydroxy-cyclopentyl)-propionamide trifluoroacetate

Step 1: {(R)-1-[9-((1R,2S,3R,4S)-2,3-Dihydroxy-4-propionylamino-cyclopentyl)-6-(2,2-diphenyl-ethylamino)-9H-purin-2-yl]-pyrrolidin-3-yl}-carbamic acid tert-butyl ester trifluoroacetate

A reaction mixture comprising N-{(1S,2R,3S,4R)-4-[2-chloro-6-(2,2-diphenyl-ethylamino)-purin-9-yl]-2,3-dihydroxy-cyclopentyl}-propionamide (Intermediate A) (2.5 g, 4.80 mmol) and (3R)-(+)-(3-Boc-amino)pyrrolidine (2.5 g, 13.6 mmol) in DMSO (8 ml) is heated at 100° C. overnight. The resulting mixture is purified by reverse phase column chromatography (Isolute™ C18, 0-100% MeOH in water—0.1% TFA) to yield the title product which is used in the next step.

Step 2: N-{(1S,2R,3S,4R)-4-[2-((R)-3-amino-pyrrolidin-1-yl)-6-(2,2-diphenyl-ethylamino)-purin-9-yl]-2,3-dihydroxy-cyclopentyl}-propionamide

{(R)-1-[9-((1R,2S,3R,4S)-2,3-Dihydroxy-4-propionylamino-cyclopentyl)-6-(2,2-diphenyl-ethylamino)-9H-purin-2-yl]-pyrrolidin-3-yl}-carbamic acid tert-butyl ester trifluoroacetate (step 1) (3.22 g, 4.80 mmol) is dissolved in 1.25 M HCl in MeOH (60 ml, 75 mmol) and left to stir at room temperature overnight. The solvent is removed in vacuo and the crude product is dissolved in a minimal volume of EtOH/saturated sodium carbonate solution and purified by reverse phase column chromatography (Isolute™ C18, 0-100% MeOH in water) to yield the title product.

Step 3: N-((1S,2R,3S,4R)-4-{6-(2,2-Diphenyl-ethylamino)-2-[(R)-3-(3-pyridin-2-ylmethyl-ureido)-pyrrolidin-1-yl]-purin-9-yl}-2,3-dihydroxy-cyclopentyl)-propionamide trifluoroacetate

A suspension of N-{(1S,2R,3S,4R)-4-[2-((R)-3-amino-pyrrolidin-1-yl)-6-(2,2-diphenyl-ethylamino)-purin-9-yl]-2,3-dihydroxy-cyclopentyl}-propionamide (step 2) (0.12 mg, 230 μmol) and sodium hydrogencarbonate (27 mg, 253 μmol) in DMSO (300 μl) is treated with phenyl chloroformate (36 mg, 230 μmol) and then stirred at room temperature for 3 hours. This reaction mixture is added to 2-picolylamine (4.1 mg, 38 μmol) and stirred at 80° C. for 5 hours. Purification of the crude product by C-18 reverse phase column chromatography eluting with acetonitrile:water:TWA (0.1%) (gradient of 0 to 100% acetonitrile) to yield the title compound. [M+H]⁺ 705.

### Example 4

### N-[(1S,2R,3S,4R)-4-(6-((S)-1-Benzyl-2-hydroxy-ethylamino)-2-{(R)-3-[3-(4-sulfamoyl-phenyl)-ureido]-pyrrolidin-1-yl}-purin-9-yl)-2,3-dihydroxy-cyclopentyl]-2-hydroxy-acetamide

A mixture comprising N-{(1S,2R,3S,4R)-4-[6-((S)-1-benzyl-2-hydroxy-ethylamino)-2-chloro-purin-9-yl]-2,3-dihydroxy-cyclopentyl}-2-hydroxy-acetamide (Intermediate B) (200 mg) and 3-((R)-3-pyrrolidin-3-ylureido)-benzenesulfonamide (Intermediate E) (480 mg) in DMSO (0.4 ml) is heated at 80° C. for 6 hours. The reaction mixture is purified by C-18 reverse phase column chromatography eluting with acetonitrile:water:NH₃ (0.1) (gradient of 0 to 100% acetonitrile) to yield the title compound. [M+H]⁺ 647.

### Example 5

### [(1S,2R,3S,4R)-4-(6-(2,2-Diphenyl-ethylamino)-2-{(R)-3-[3-(3-sulfamoyl-phenyl)-ureido]-pyrrolidin-1-yl}-purin-9-yl)-2,3-dihydroxy-cyclopentyl]-carbamic acid methyl ester

This compound is prepared analogously to N-[(1S,2R,3S,4R)-4-(6-((S)-1-Benzyl-2-hydroxy-ethylamino)-2-{(R)-3-[3-(4-sulfamoyl-phenyl)-ureido]-pyrrolidin-1-yl}-purin-9-yl)-2,3-dihydroxy-cyclopentyl]-2-hydroxy-acetamide (Example 4) by replacing N-{(1S,2R,3S,4R)-4-[6-((S)-1-Benzyl-2-hydroxy-ethylamino)-2-chloro-purin-9-yl]-2,3-dihydroxy-cyclopentyl}-2-hydroxy-acetamide (Intermediate B) with {(1S,2R,3S,4R)-4-[2-Chloro-6-(2,2-diphenyl-ethylamino)-purin-9-yl]-2,3-dihydroxy-cyclopentyl}-carbamic acid methyl ester (Intermediate F) and by replacing 3-((R)-3-pyrrolidin-3-ylureido)-benzenesulfonamide (Intermediate E) with 1-pyridin-3-yl-3-(R)-pyrrolidin-3-yl-urea (Intermediate D). [M+H]⁺ 771.

### Example 6

### N,N′-(1S,1S′,2R,2R′,3S,3S′,4R,4R′)-4,4′-((S)-2,2′-((3R3′R)-3,3′-carbonylbis(azanediyl)bis(pyrrolidine-3,1-diyl))bis(6-((S)-1-hydroxy-3-phenylpropan-2-ylamino)-9H-purine-9,2diyl))bis(2,3-dihydroxycyclopentane-4,1-diyl)bis(2-hydroxyacetamide)

A solution of N-{(1S,2R,3S,4R)-4-[6-((S)-1-benzyl-2-hydroxy-ethylamino)-2-chloro-purin-9-yl]-2,3-dihydroxy-cyclopentyl}-2-hydroxy-acetamide (Intermediate B) (140 mg, 0.29 mmol) and 1,3-di(R)-pyrrolidin-3-yl-urea (Intermediate G) (70 mg, 0.35 mmol) in DMSO (0.3 ml) is treated with anhydrous sodium carbonate (49 mg) and heated at 100° C. overnight. Purification of the resulting mixture by C-18 reverse phase column chromatography eluting with acetonitrile:water:TFA (0.1%) (gradient of 0 to 100% acetonitrile) to yields the title compound. [M+2H]²⁺ 540

### Example 7

### N,N′-(1S,1S′,2R,2R′,3S,3S′,4R,4R′)-4,4′-(6,6′-(1R,4R)-cyclohexane-1,4-diylbis(azanediyl)bis(2-(2-(1-methyl-1Himidazol-4-yl)ethylamino)-9H-purine-9,6-diyl))bis(2,3-dihydroxycyclopentane-4,1-diyl)bis(2-hydroxyacetamide)

Step 1:

(1S,2R,3S,5R)-3-(Di-Boc-amino)-5-(2,6-dichloro-purin-9-yl)-cyclopentane-1,2-diol (Step A4) (0.5 g, 0.992 mmol) in IPA (5 ml) is treated with diamine (trans-1,4)cyclohexane (56.6 mg, 0.446 mmol) and DIPEA (0.432 ml, 2.48 mmol). The suspension is heated at 83° C. overnight and after cooling to room temperature, the solvent is removed in vacuo. The resulting solid is triturated with water/MeOH to afford the product as a beige solid. [M+H]⁺ 1049/1052.

Step 2:

The product from step 1 (0.2932 g, 0.279 mmol) is dissolved in MeOH (5 ml) and treated with 4M HCl in dioxane (3 ml). The resulting orange mixture is room temperature for 2 hours and then concentrated in vacuo to afford the desired product as a hydrochloride salt. [M+H]⁺ 651.

Step 3:

The product from step 2 (0.1 g, 0.119 mmol) in THF (1 ml) and MeOH (1 ml) is treated with TEA (0.25 ml, 1.78 mmol) and stirred at room temperature for 1 hour. Acetoxy acetylchloride (0.0.384 ml, 0.714 mmol) is then added and stirring continued for 14 days. The solvent is removed in vacuo and the resulting residue is treated with MeOH and potassium carbonate (20 mg) in water (0.5 ml). The mixture is stirred at room temperature overnight and then purification is carried out by reverse phase column chromatography (Isolute™ C18, 100% water followed by 100% MeOH) to yield desired product. [M+H]⁺ 765/767.

Step 4:

This compound is prepared from the product of Step 3 and C-(1-methyl-1H-imidazol-4-yl)-methylamine analogously to Example 6. Potassium carbonate is used in place of anhydrous sodium carbonate. [M+2H]²⁺ 472.

Intermediate A

### N-{(1S,2R,3S,4R)-4-[2-Chloro-6-(2,2-diphenyl-ethylamino)-purin-9-yl]-2,3-dihydroxy-cyclopentyl}-propionamide

Step A1: (1S,4R)-4-(2,6-Dichloro-purin-9-yl)-cyclopent-2-enol 2,6-Dichloropurine (10 g, 52.90 mmol), (1S,4R)-cis 4-acetoxy-2-cyclopenten-1-ol (10 g. 70.40 mmol), tris(dibenzylideneacetone)dipalladium(0) (3.20 g, 3.50 mmol) and polymer supported triphenylphosphine (3 mmol/g, 11.60 g, 35.00 mmol) are placed in an oven-dried flask under an atmosphere of argon. Dry deoxygenated THF (80 ml) is added and the reaction mixture is stirred gently for 5 minutes. Triethylamine (20 ml) is added and the reaction mixture is stirred at 50° C. The reaction is shown to be complete by LCMS after 1 hour. The reaction mixture is allowed to cool, filtered and the solvent is removed in vacuo. The title compound is obtained after purification by flash column chromatography (silica, dichloromethane/methanol 25:1). ¹H nmr (CDCl₃, 400 MHz); 8.30 (s, 1H), 6.40 (m, 1H), 5.90 (m, 1H), 5.50 (m, 1H), 4.95 (m, 1H), 3.05 (m, 1H), 2.10 (m, 1H), [M+H]⁺ 271.

Step A2: Carbonic Acid (1S,4R)-4-(2,6-dichloro-purin-9-yl)-cyclopent-2-enyl Ester Ethyl Ester

(1S,4R)-4-(2,6-Dichloro-purin-9-yl)-cyclopent-2-enol (9.5 g, 35.05 mmol) is placed in an oven-dried flask under an atmosphere of argon. D THF (200 mL) is added followed by dr pyridine (5.54 g, 70.1 mmol). Ethyl chloroformate (15.21 g, 140.2 mmol) is added slowly so that the temperature does not rise above 40° C. and the reaction mixture is stirred at room temperature. The reaction is shown to be complete by LCMS after 1 hour. The solvent is removed in vacuo and the residue is partitioned between dichloromethane (200 mL) and water (200 mL). The organic layer is washed with water (150 ml) and brine (150 ml), dried over MgSO₄, filtered and the solvent is removed in vacuo. The title compound is obtained after crystallisation from methanol. ¹H nmr (CDCl₃, 400 MHz); 8.20 (s, 1H), 6.45 (m, 1H), 6.25 (m, 1H), 5.75 (m, 1H), 5.70 (m, 1H), 4.25 (q, 2H), 3.20 (m, 1H), 2.05 (m, 1H), 1.35 (t, 3H), [M+H]⁺ 343.

Step A3: Di-Boc-[(1S,4R)-4-(2,6-dichloro-purin-9-yl)-cyclopent-2-enyl]-amine

Carbonic acid (1S,4R)-4-(2,6-dichloro-purin-9-yl)-cyclopent-2-enyl ester ethyl ester (2.5 g, 7.29 mmol), di-t-butyliminodicarboxylate (1.74 g, 8.02 mmol), and triphenylphosphine (0.29 g, 1.09 mmol) are placed in an oven-dried flask under an atmosphere of argon. Dry deoxygenated THF (30 ml) is added followed by tris(dibenzylideneacetone)dipalladium(0) (0.33 g, 0.36 mmol) and the reaction mixture is stirred at room temperature. The reaction is shown to be complete by LCMS after 3 hours. The solvent is removed in vacuo and the title compound is obtained after purification by flash column chromatography (silica, ethyl acetate/iso-hexane 4:1) ¹H nmr (CDCl₃, 400 MHz); 8.70 (s, 1H), 6.20 (m, 1H), 5.85 (m, 1H), 5.80 (m, 1H), 5.40 (m, 1H), 3.20 (m, 1H), 2.15 (m, 1H), 1.55 (s, 1H), [M+H]⁺ 470.

Step A4: (1S,2R,3S,5R)-3-(Di-Boc-amino)-5-(2,6-dichloro-purin-9-yl)-cyclopentane-1,2-diol

A mixture comprising di-Boc-[(1S,4R)-4-(2,6-dichloro-purin-9-yl)-cyclopent-2-enyl]-amine (1.30 g, 2.77 mmol) (1.49 g, 3.17 mmol), methane sulphonamide (0.30 g, 3.17 mmol) and AD-mix-α (6.75 g, 1.5 g/mmol) in t-butanol/water (20 ml of a 1:1 mixture) is treated with osmium tetroxide (1.5 ml, 4% w/w in water). After stirring vigorously at room temperature overnight, the reaction mixture is partitioned between EtOAc and water. The organic portion is separated, washed with water, brine, dried (MgSO₄) and concentrated in vacuo to yield the title compound which is used in the next step without further purification. ¹H nmr (CDCl₃, 400 MHz); 8.35 (s, 1H), 4.80 (m, 1H), 4.70 (m, 1H), 4.50 (m, 1H), 3.85 (m, 1H), 3.75 (m, 1H), 3.10 (m, 1H), 2.75 (m, 1H), 2.55 (m, 1H), 1.55 (s, 18H), [M+H]⁺ 504.

Step A5: (1S,2R,3S,5R)-3-Amino-5-(2,6-dichloro-purin-9-yl)-cyclopentane-1,2-diol trifluoroacetate

A solution of (1S,2R,3S,5R)-3-(di-Boc-amino)-5-(2,6-dichloro-purin-9-yl)-cyclopentane-1,2-diol (0.55 g, 1.09 mmol) in DCM (4 ml) is treated with TFA (2 ml) and stirred at room temperature. After 2 hours, the solvent is removed in vacuo to yield the title compound which is used in the next step without further purification. [M+H]⁺ 304.

Step A6: N-[(1S,2R,3S,4R)-4-(2,6-Dichloro-purin-9-yl)-2,3-dihydroxy-cyclopentyl]-propionamide

A solution of (1S,2R,3S,5R)-3-amino-5-(2,6-dichloro-purin-9-yl)-cyclopentane-1,2-diol trifluoroacetate (0.304 g, 1.0 mmol) in THF (10 ml) is treated with DIPEA (0.387 g, 3.0 mmol) followed by propionyl chloride (0.093 g, 1.0 mmol). After stirring at room temperature for 2 hours, the solvent is removed in vacuo and the title compound is obtained after purification by reverse phase column chromatography (Isolute™ C18, 0-100% acetonitrile in water—0.1% TFA). [M+H]⁺ 360.

Step A7: N-{(1S,2R,3S,4R)-4-[2-Chloro-6-(2,2-diphenyl-ethylamino)-purin-9-yl]-2,3-dihydroxy-cyclopentyl}-propionamide

N-[(1S,2R,3S,4R)-4-(2,6-dichloro-purin-9-yl)-2,3-dihydroxy-cyclopentyl]-propionamide (160 mg, 0.44 mmol) (Step A6) is dissolved in THF (5 ml) under an atmosphere of argon. DIPEA (69 mg, 0.53 mmol) is added followed by 2,2-diphenylethylamine (96 mg, 0.49 mmol) and the reaction mixture is stirred at 50° C. The reaction is shown to be complete by LCMS after 2 hours. The solvent is removed in vacuo and the title compound is obtained after purification by reverse phase column chromatography (Isolute™ C18, 0-100% acetonitrile in water—0.1% TFA)). ¹H nmr (MeOD, 400 MHz); 8.00 (s, 1H), 7.40-7.15 (m, 10H), 4.75 (m, 1H), 4.60 (m, 1H), 4.50 (m, 1H), 4.20 (m, 3H), 3.95 (m, 1H), 2.85 (m, 1H), 2.40 (q, 2H), 2.10 (m, 1H), 1.20 (t, 3H), [M+H]⁺ 521.

Intermediate A may also be prepared using the following process:

AA1: {2-Chloro-9-[(1R,4S)-4-(di-Boc-amino)-cyclopent-2-enyl]-9H-purin-6-yl}-(2,2-diphenyl-ethyl)-amine

(1S,2R,3S,5R)-3-(Di-Boc-amino)-5-(2,6-dichloro-purin-9-yl)-cyclopentane-1,2-diol (prepared according to the procedure illustrated on page 55 Example 4, step 4 of WO 2006/045552) (13.0 g, 27.66 mmol) is dissolved in THY (250 ml) under an atmosphere of argon. Diisopropylamine (4.28 g, 33.19 mmol) is added followed by 2,2-diphenylethylamine (6.0 g, 30.43 mmol) and the reaction mixture is stirred at 50° C. The reaction is shown to be complete by LCMS after 18 hours. The solvent is removed in vacuo and the reaction mixture is partitioned between dichloromethane (250 ml) and 0.1M HCl (250 ml). The organic layer is washed with water (200 ml) and brine (200 ml), dried over MgSO₄, filtered and the solvent is removed in vacuo to give the title compound. ¹H nmr (CDCl₃, 400 MHz); 8.05 (s, 1H), 7.30-7.10 (m, 10H), 6.00 (m, 1H), 5.70 (m, 2H), 5.60 (m, 1H), 5.20 (m, 1H), 4.30 (m, 1H), 4.20 (m, 1H), 3.65 (m, 1H), 3.05 (m, 1H), 2.00 (m, 1H), 1.70 (m, 1H), 1.40 (s, 18H), [M+H]⁺ 631.

AA2: (1R,2S,3R,5S)-3-[2-Chloro-6-(2,2-diphenyl-ethylamino)-purin-9-yl]-5-(di-Boc-amino)-cyclopentane-1,2-diol

A solution of {2-Chloro-9-[(1R,4S)-4-(di-Boc-amino)-cyclopent-2-enyl]-9H-purin-6-yl}-(2,2-diphenyl-ethyl)-amine (2.9 g, 4.6 mmol) in THF (60 ml) is treated with 4-methyl morpholine N-oxide (1.1 g, 9.3 mmol) and osmium tetroxide (4% solution in water) (6 ml) and the mixture is stirred at room temperature for 48 hours. The solvent is removed under reduced pressure and the residue is purified by column chromatography on silica gel eluting with a gradient system of methanol:dichloromethane (0:100 by volume) gradually changing to methanol:dichloromethane (4:96 by volume) to afford the title compound. [M+H]⁺ 665.34

AA3: (1S,2R,3S,5R)-3-Amino-5-[2-chloro-6-(2,2-diphenyl-ethylamino)-purin-9-yl]-cyclopentane-1,2-diol trifluoroacetate

(1R,2S,3R,5S)-3-[2-Chloro-6-(2,2-diphenyl-ethylamino)-purin-9-yl]-5-(di-Boc-amino)-cyclopentane-1,2-diol (10.3 g, 15.50 mmol) is dissolved in dichloromethane (50 ml). TEA (25 ml) is added and the reaction mixture is stirred at room temperature. The reaction is shown to be complete by LCMS after 2 hours. The solvent is removed in vacuo to give the title compound. ¹H nmr (MeOD, 400 MHz); 7.90 (s, 1H), 7.30-7.10 (m, 10H), 4.65 (m, 1H), 4.50 (m, 1H), 4.40 (m, 1H), 4.20 (m, 1H), 4.10 (m, 2H), 3.50 (m, 1H), 2.75 (m, 1H), 2.15 (m, 1H), [M+H]⁺ 465.

AA4: N-{(1S,2R,3S,4R)-4-[2-Chloro-6-(2,2-diphenyl-ethylamino)-purin-9-yl]-2,3-dihydroxy-cyclopentyl}-propionamide

(1S,2R,3S,4R)-3-Amino-5-[2-chloro-6-(2,2-diphenyl-ethylamino)-purin-9-yl]-cyclopentane-1,2-diol trifluoroacetate (9.50 g, 16.42 mmol) and diisopropylethylamine (6.36 g, 49.27 mmol) are placed in a flask with dry THF (150 ml). Propionyl chloride (1.52 g, 16.42 mmol) is added dropwise and the reaction mixture is stirred at room temperature. The reaction is shown to be complete by LCMS after 1 hour. The solvent is removed in vacuo and the residue is partitioned between dichloromethane (250 ml) and water (250 ml). The organic layer is washed with water (200 ml) and brine (200 ml), dried over MgSO₄, filtered and the solvent is removed in vacuo. The solid is recrystallised from 1,2-dichloroethane to give the title compound. ¹H nmr (MeOD, 400 MHz); 8.00 (s, 1H), 7.40-7.15 (m, 10H), 4.75 (m, 1H), 4.60 (m, 1H), 4.50 (m, 1H), 4.20 (m, 3H), 3.95 (m, 1H), 2.85 (m, 1H), 2.40 (q, 2H), 2.10 (m, 1H), 1.20 (t, 3H), [M+H]⁺ 521.

Intermediate B

### N-{(1S,2R,3S,4R)-4-[6-((S)-1-Benzyl-2-hydroxy-ethylamino)-2-chloro-purin-9-yl]-2,3-dihydroxy-cyclopentyl}-2-hydroxy-acetamide

B1

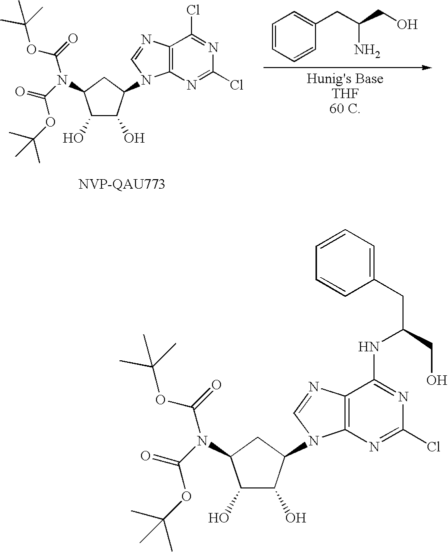

This compound is prepared analogously to N-{(1S,2R,3S,4R)-4-[2-Chloro-6-(2,2-diphenyl-ethylamino)-purin-9-yl]-2,3-dihydroxy-cyclopentyl}-propionamide (Intermediate A) by replacing N-[(1S,2R,3S,4R)-4-(2,6-dichloro-purin-9-yl)-2,3-dihydroxy-cyclopentyl]-propionamide (Intermediate A6) (Step A7) with (1S,2R,3S,5R)-3-(di-Boc-amino)-5-(2,6-dichloro-purin-9-yl)-cyclopentane-1,2-diol (Intermediate A4) and by replacing 2,2-diphenylethylamine (Step A7) with (4Z,6Z)-(S)-phenylalinol. [M+H]⁺ 619.

B2: (1S,2R,3S,5R)-3-Amino-5-[6-((S)-1-benzyl-2-hydroxy-ethylamino)-2-chloro-purin-9-yl]-cyclopentane-1,2-diol hydrochloride

The product of Step B1 (409 mg, 0.62 mmol) is dissolved in MeOH (3 ml) and 4M HCl in dioxane (2 ml). The reaction mixture is stirred at room temperature for 3 hours and then concentrated in vacuo to afford the title product which is used in the next step without further purification. [M+H]+ 419.

B3: Acetic acid {(1S,2R,3S,4R)-4-[6-((S)-1-benzyl-2-hydroxy-ethylamino)-2-chloro-purin-9-yl]-2,3-dihydroxy-cyclopentylcarbamoyl}-methyl ester

1S,2R,3S,5R)-3-Amino-5-[6-((S)-1-benzyl-2-hydroxy-ethylamino)-2-chloro-purin-9-yl]-cyclopentane-1,2-diol hydrochloride (Intermediate B2) (320 mg, 0.7 mmol) is dissolved in THF (3 ml) and treated with TEA (0.98 ml) and acetoxyacetylchloride (79 μl, 0.74 mmol). The reaction mixture is stirred at room temperature for 1 hour. The solvent is removed in vacuo and the title compound is obtained after purification by reverse phase column chromatography (Isolute™ C18, 0-100% acetonitrile in water—0.1% TFA). [M+H]+ 119.

B4: N-{(1S,2R,3S,4R)-4-[6-((S)-1-Benzyl-2-hydroxy-ethylamino)-2-chloro-purin-9-yl]-2,3-dihydroxy-cyclopentyl}-2-hydroxy-acetamide

Acetic acid {(1S,2R,3S,4R)-4-[6-((S)-1-benzyl-2-hydroxy-ethylamino)-2-chloro-purin-9-yl]-2,3-dihydroxy-cyclopentylcarbamoyl}-methyl ester (Intermediate B3) (122 mg, 0.19 mmol) is dissolved in MeOH (4 ml) and treated with potassium carbonate (53 mg). The reaction mixture is stirred at room temperature for 48 hours and then concentrated in vacuo to afford the title product which is used without further purification. [M+H]+ 477

Intermediate C

### {(1S,2R,3S,4R)-4-[2-((R)-3-Amino-pyrrolidin-1-yl)-6-(2,2-diphenyl-ethylamino)-purin-9-yl]-2,3-dihydroxy-cyclopentyl}-carbamic acid benzyl ester

C1: Dibenzyl Iminodicarbonate

A cooled (0° C.) solution of benzyl carbamate (4.0 g, 27 mmol) in THF (100 ml) under an inert atmosphere of argon is treated with potassium hydride (3.2 g of a 35% w/w dispersion in oil, 28 mmol) portionwise over 10 minutes. The reaction mixture is allowed to warm to room temperature over 30 minutes after which time benzyl chloroformate (5.0 g, 29 mmol) is added. After stirring at room temperature for 2 hours, the reaction is quenched with water (20 ml). The THF is removed in vacuo and the resulting mixture is partitioned between EtOAc and 2M HCl. The organic portion is separated and washed with brine, dried (MgSO₄) and concentrated in vacuo. The resulting oil is purified by chromatography on silica eluting with 1:3 EtOAc/iso-hexane to yield a product which is recrystallised from DCM/iso-hexane to afford the title product. [M+H]⁺ 286.

C2: Preparation of Intermediate C2

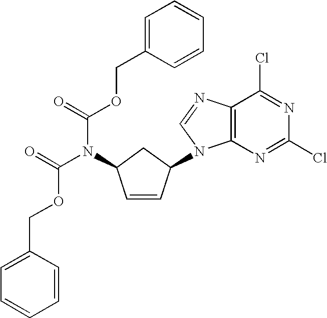

A solution comprising carbonic acid (1S,4R)-4-(2,6-dichloro-purin-9-yl)-cyclopent-2-enyl ester ethyl ester (prepared according to the procedure illustrated on page 54 Example 4, step 2 of WO 2006/045552) (2.0 g, 5.83 mmol), dibenzyl iminodicarbonate (Intermediate C1) (2.2 g, 7.58 mmol) and triphenyl phosphine (229 mg, 0.9 mmol) in THF (20 ml) is stirred at room temperature for 30 minutes. Tris(dibenzylideneacetone)dipalladium (0) (238 mg, 0.3 mmol) is added and the resulting mixture is stirred at room temperature for 1.5 hours. The solvent is removed in vacuo and the crude product is purified by chromatography on silica eluting with MeOH/DCM (gradient of 0 to 1% MeOH) to yield the title compounds [M+H]⁺ 538.

C3: Preparation of Intermediate C3

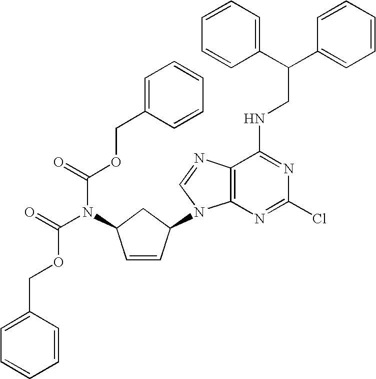

This compound is prepared analogously to 2-chloro-9-[(1R,4S)-4-(di-Boc-amino)-cyclopent-2-enyl]-9H-purin-6-yl}-(2,2-diphenyl-ethyl)-amine (AA1) by replacing (1S,2R,3S,5R)-3-(Di-Boc-amino)-5-(2,6-dichloro-purin-9-yl)-cyclopentane-1,2-diol (Intermediate A4) with Intermediate C2. [M+H]⁺ 699.

C4: Preparation of Intermediate C4

This compound is prepared analogously to (1R,2S,3R,5S)-3-[2-chloro-6-(2,2-diphenyl-ethylamino)-purin-9-yl]-5-(di-Boc-amino)-cyclopentane-1,2-diol (AA2) by replacing {2-Chloro-9-[(1R,4S)-4-(di-Boc-amino)-cyclopent-2-enyl]-9H-purin-6-yl}-(2,2-diphenyl-ethyl)-amine with Intermediate C3. [M+H]⁺ 733.

C5: {(R)-1-[9-((1R,2S,3R,4S)-4-Benzyloxycarbonylamino-2,3-dihydroxy-cyclopentyl)-6-(2,2-diphenyl-ethylamino)-9H-purin-2-yl]-pyrrolidin-3-yl}-carbamic acid tert-butyl ester

A suspension of Intermediate C4 (1.03 g, 1.4 mmol) and (3R)-(+)-3-(Boc-amino)pyrrolidine (1.03 g, 5.5 mmol) in acetonitrile (2 ml) is treated with sodium iodide (ca. 2 mg) and then heated using microwave radiation in a Personal Chemistry Emrys™ Optimizer microwave reactor at 160° C. for 1 hour. The solvent is removed in vacuo and the crude residue is partioned between DCM and 0.2 M HCl. The organic layer is separated and the aqueous portion is extracted with DCM. The combined organic extracts are washed with saturated sodium bicarbonate solution, water, brine, dried (MgSO₄) and concentrated in vacuo to afford the title compound as a brown oil. [M+H]⁺ 745.

C6: {(1S,2R,3S,4R)-4-[2-((R)-3-amino-pyrrolidin-1-yl)-6-(2,2-diphenyl-ethylamino)-purin-9-yl]-2,3-dihydroxy-cyclopentyl}-carbamic acid benzyl ester

A solution of {(R)-1-[9-((1R,2S,3R,4S)-4-benzyloxycarbonylamino-2,3-dihydroxy-cyclopentyl)-6-(2,2-diphenyl-ethylamino)-9H-purin-2-yl]-pyrrolidin-3-yl}-carbamic acid tert-butyl ester (Intermediate C5) (1.24 g, 1.7 mmol) in MeOH (3 ml) is treated with 4M HCl in dioxane (5 ml) and stirred at room temperature for 2 hours. The solvent is removed in vacuo and purification is carried out by reverse phase column chromatography (Isolute™ C18, 0-100% acetonitrile in water—0.1% HCl). The fractions are collected and the MeCN is removed in vacuo. The remaining aqueous portion is basified with saturated sodium bicarbonate solution and extracted with DCM. The combined organic extracted are dried (MgSO₄) and concentrated in vacuo to afford the title product. [M+H]⁺ 649.

Intermediate D

### 3-((R)-3-Pyrrolidin-3-ylureido)-benzenesulfonamide

D1: (3-Sulfamoyl-phenyl)-carbamic Acid Phenyl Ester

A suspension of phenyl chloroformate (3.64 ml, 29 mmol) in DCM (20 ml) and pyridine (10 ml) is cooled to 0° C. and then treated dropwise with a solution of 3-amino-benzenesulfonamide (5 g, 29 mmol) in DCM (10 ml) and pyridine (20 ml). The mixture is stirred and allowed to warm to room temperature overnight. The solvent is removed in vacuo and the resulting oil is dissolved in 1M HCl and DCM. A white solid precipitates and is collected by filtration and washed with water. The solid is dried in vacuo to afford the title compound. [M+H]⁺ 293.

D2: 3-[3-((R)-1-Benzyl-pyrrolidin-3-yl)-ureido]-benzenesulfonamide A solution of (R)—N-benzyl-3-aminopyrrolidine (14.9 g, 0.084 mol) in methanol (100 mL) is added to a suspension of (3-sulfamoyl-phenyl)-carbamic acid phenyl ester (Intermediate D1) (25 g, 0.084 mol). The resulting pale orange solution is stirred at reflux for two hours, then allowed to cool to room temperature, before the volatile components are removed under reduced pressure. The orange syrup is purified by flash column chromatography (silica; DCM/methanol 10:1) to give a beige foamed solid which is used in the next step.

D3: 3-((R)-3-Pyrrolidin-3-ylureido)-benzenesulfonamide

A solution of 3-[3-((R)-1-benzyl-pyrrolidin-3-yl)-ureido]-benzenesulfonamide (Intermediate D2) (25 g, 0.067 mol) in ethanol (250 mL) is purged with nitrogen, and palladium hydroxide (2.5 g, 20% w/w) is added. The suspension is purged with hydrogen and stirred under a positive pressure of hydrogen for 24 hours. Filtration though Celite® (filter material) and removal of solvent under reduced pressure gives the product as a colourless waxy solid. [M+H]⁺ 285.

Intermediate E

### 1-Pyridin-3-yl-3-(R)-pyrrolidin-3-yl-urea

This compound is prepared analogously to Intermediate D by replacing 3-amino-benzenesulfonamide with 4-amino-benzenesulfonamide. [M+H]⁺ 285.

Intermediate F

### {(1S,2R,3S,4R)-4-[2-Chloro-6-(2,2-diphenyl-ethylamino)-purin-9-yl]-2,3-dihydroxy-cyclopentyl}-carbamic acid methyl ester

This compound is prepared analogously to N-[(1S,2R,3S,4R)-4-(2,6-Dichloro-purin-9-yl)-2,3-dihydroxy-cyclopentyl]-propionamide (Intermediate A6) by replacing propionyl chloride with methyl chloroformate. [M+H]⁺ 523.

Intermediate G

### 1,3-Di(R)-pyrrolidin-3-yl-urea

G1: 1,3-Bis-((R)-1-benzyl-pyrrolidin-3-yl)-urea

A solution comprising (R)-1-benzyl-pyrrolidin-3-ylamine (5.0 g, 28.4 mmol) in DCM (10 ml) is treated with CDI (2.3 g, 14.2 mmol) and the reaction mixture is stirred at room temperature for 48 hours. The solvent is removed in vacuo and the resulting residue is dissolved in ethyl acetate. This portion is washed with water followed by brine, dried (MgSO₄) and concentrated in vacuo to yield the title compound as pale orange solid. The solid is used in the next step without further purification.

G2: 1,3-Di(R)-pyrrolidin-3-yl-urea

To a solution of 1,3-bis-((R)-1-benzyl-pyrrolidin-3-yl)-urea (Intermediate G1) (5.34 g, 14.1 mmol) in ethanol (80 ml) under an inert atmosphere of argon is added palladium hydroxide on carbon (1.07 g). The reaction mixture is purged with argon and placed under an atmosphere of hydrogen for two days after which time, the mixture is filtered and the catalyst washed with ethanol. The organic portions are combined and concentrated in vacuo to yield the title compound as a white solid. [M+H]⁺ 199.

1. A composition comprising, separately or together
(A) a compound selected from:
((1S,2R,3S,4R)-4-{6-(2,2-Diphenyl-ethylamino)-2-[(S)-3-(3-pyridin-3-yl-ureido)-pyrrolidin-1-yl]-purin-9-yl}-2,3-dihydroxy-cyclopentyl)-carbamic acid methyl ester;
((1S,2R,3S,4R)-4-{6-(2,2-Diphenyl-ethylamino)-2-[(R)-3-(3-pyridin-3-yl-ureido)-pyrrolidin-1-yl]-purin-9-yl}-2,3-dihydroxy-cyclopentyl)-carbamic acid methyl ester;
Cyclopropanecarboxylic acid ((1S,2R,3S,4R)-4-{6-(2,2-diphenyl-ethylamino)-2-[(R)-3-(3-pyridin-3-yl-ureido)-pyrrolidin-1-yl]-purin-9-yl}-2,3-dihydroxy-cyclopentyl)-amide;
N-((1S,2R,3S,4R)-4-{6-(2,2-Diphenyl-ethylamino)-2-[(R)-3-(3-pyridin-2-ylmethyl-ureido)-pyrrolidin-1-yl]-purin-9-yl}-2,3-dihydroxy-cyclopentyl)-propionamide trifluoroacetate;
N-[(1S,2R,3S,4R)-4-(6-((S)-1-Benzyl-2-hydroxy-ethylamino)-2-{(R)-3-[3-(4-sulfamoyl-phenyl)-ureido]-pyrrolidin-1-yl}-purin-9-yl)-2,3-dihydroxy-cyclopentyl]-2-hydroxy-acetamide;
[(1S,2R,3S,4R)-4-(6-(2,2-Diphenyl-ethylamino)-2-{(R)-3-[3-(3-sulfamoyl-phenyl)-ureido]-pyrrolidin-1-yl}-purin-9-yl)-2,3-dihydroxy-cyclopentyl]-carbamic acid methyl ester;
N,N′-(1S,1S′,2R,2R′,3S,3S′,4R,4R′)-4,4′-((S)-2,2′-((3R3′R)-3,3′-carbonylbis(azaniediyl)bis(pyrrolidine-3,1-diyl))bis(6-((S)-1-hydroxy-3-phenylpropan-2-ylamino)-9H-purine-9,2diyl))bis(2,3-dihydroxycyclopentane-4,1-diyl)bis(2-hydroxyacetamide); and
N,N-(1S,1S′,2R,2R′,3S,3S′,4R,4R′)-4,4′-(6,6′-(1R,4R)-cyclohexane-1,4-diylbis(azanediyl)bis(2-(2-(1-methyl-1Himidazol-4-yl)ethylamino)-9H-purine-9,6-diyl))bis(2,3-dihydroxycyclopentane-4,1-diyl)bis(2-hydroxyacetamide), in free or salt or solvate form; and
(B) one or more compounds selected from:
(i) a corticosteroid,
(ii) a beta-2 adrenoceptor agonist,
(iii) an antimuscarinic agent,
(iv) an A_{2B} antagonist,
(v) an antihistamine,
(vi) a caspase inhibitor,
(vii) an ENaC inhibitor,
(viii) an LTB4 antagonist,
(ix) an LTD4 antagonist,
(x) a serine protease inhibitor,
(xi) a PDE4 inhibitor and
(xi) a dual-acting beta-2 adrenoceptor agonist/muscarinic antagonist;
for simultaneous, sequential or separate administration in the treatment of an inflammatory or obstructive airways disease.

2. A composition according to claim 1, wherein (B) is selected from a corticosteroid, a beta-2 adrenoceptor agonist and an antimuscarinic agent.

3. A composition according to claim 1, wherein (B) is selected from formoterol, indacaterol, glycopyrolate, (R)-3-(2-Hydroxy-2,2-diphenyl-acetoxy)-1-(isoxazol-3-ylcarbamoylmethyl)-1-azonia-bicyclo[2.2.2]octane and (R)-3-((R)-2-Cyclohexyl-2-hydroxy-2-phenyl-acetoxy)-1-(isoxazol-3-ylcarbamoylmethyl)-1-azonia-bicyclo[2.2.2]octane.

4. A composition according to claim 1, wherein (A) is ((1S,2R,3S,4R)-4-{6-(2,2-Diphenyl-ethylamino)-2-[(R)-3-(3-pyridin-3-yl-ureido)-pyrrolidin-1-yl]-purin-9-yl}-2,3-dihydroxy-cyclopentyl)-carbamic acid methyl ester or a salt or solvate thereof.

5. A composition comprising a mixture of effective amounts of (A) as defined in claim 1 and (B) as defined in claim 1, optionally together with at least one pharmaceutically acceptable carrier.

6. Use of (A) as defined in claim 1 and (B) as defined in claim 1 in the preparation of a medicament for combination therapy by simultaneous, sequential or separate administration of (A) and (B) in the treatment of an inflammatory or obstructive airways disease.

7. A kit comprising a composition according to claim 5 and an inhalational device for the administration of the composition.

8. A kit comprising (A) as defined in claim 1 and (B) as defined in claim 1 in separate unit dosage forms, said forms being suitable for administration of (A) and (B) in effective amounts; and one or more inhalation devices for administration of (A) and (B).

9. A method of treating an inflammatory or obstructive airways disease that comprises administering simultaneously, sequentially or separately to a subject in need of such treatment effective amounts of (A) as defined in claim 1 and (B) as defined in claim 1.
